SEQUENCE LISTING (38 sequences)

SEQ ID NO: 1 - protein, 4 aa, Artificial
  Binding site sequence
EDXD

SEQ ID NO: 2 - protein, 8 aa, Artificial
  Binding site sequence
EDXDAAXX

SEQ ID NO: 3 - protein, 8 aa, Artificial
  Binding site sequence
EDYDAADD

SEQ ID NO: 4 - protein, 8 aa, Artificial
  Binding site sequence
EDYDADEE

SEQ ID NO: 5 - protein, 9 aa, Homo sapiens
HYPTEHVQF

SEQ ID NO: 6 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 7 - protein, 597 aa, Homo sapiens
EVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 8 - protein, 622 aa, Mus musculus
MEPPDARAGLLWLTFLLSGYSGAQAELHVSVPPRVEVMRGEQVALDCTPREHPEHYVLEWFLVDGTGARHRLASVEPQGSEFLGTVHSLGRVPPYEVDSRGRLVIAKVQVGDGRDYVCVVKAGAAGTSEATSSVRVFATPEDTEVSPNKGTLSVMDQFAQEIATCSSNNGNPVPRITWYRNGQRLEVPMEVNQKGYITIRTVREASGLYSLTSTLYLRLHKDDRDANFHCAAHYDLPSGQHGRLDSHTFRLTLHYPTEHVEFWVGSPSTTEGWVREGDAVQLLCQGDGSPSPEYSFFRQQGTQEEQLNVNLKGNLTLERVHRNQSGIYGCRVEDYDADEEVQLVKKLKLHVAYLDPLELSVPEELFVFLNSSSTVVNCSARGLPTPTVRWTKDSVTLADGPMLSLQSVTFDSAGTYTCEASTPTVPLLSRTQSFQLIVQGAPELKPNEIMPKSGNSWTEGDEVMLTCSARGFPEPKLTWSQRGDTPAEPPFEGRGWKSSSLMVKVTSALSREGVSCEASNIHGKKGHVFHFGSVAPQTAQAGVAVMAVAVSVGLLLLVVAAFYCMRRKGRPGCCRRAEKGAPPAREPELSHSGSERPEHTGLLMGGPSGGGRGGSGGFGDEC

SEQ ID NO: 9 - protein, 624 aa, Rattus norvegicus
MEPPDARAGLLWLTLLLSGYSGAQAELHVSVPPRVEVMRGEQIALDCTPREHPENYVLEWLLVDASGARHRLASVEPQGSEFLGTIHNSRGRRPPYKIDSLGRLVIAEAQVGDERDYVCVVKAGAAGTSEATSSVRVFATPEATEVAPNKGTLSVMEQFAQEIATCSSNNGNPVPRITWYQNGQRLDVPMELNSKGYMTSRTVREASGLYSLTSTLYLRLHKEDRDASFHCAAHYDLPSGQHGRLDSHTFRLTLHYPTEHVEFWVGSPSTTEGWVREGDAVQLLCQGDGSPSPEYSFFREQGNQEEQLNVNLKGNLTLEGVHRSQSGIYGCRVEDYDADEEVQLVKKLKLHVAYLDPLELSVPEEFSVFLNSSGTVVNCSARGLPAPIVRWTKDSVTVADGPILSLDSVTFDSAGTYTCEASTPTVPLLSRTQSFQLVVQGAPELKPNEIKPKSGTSWTEGDEVMLTCSARGFPEPKLTWSQRGDTTPAEPPFEGRGWMSSSLTLKVTSALSREGVSCEASNIHGKNGHVFHFGSVAPQTAQAGVAVMAVAVSVGLLLLVVAAFYCMRRKGRPGCCQRAEKGAPPAREPELSHSGSERPEHTGLLMGGPSGGGRGGNGGFGDEC

SEQ ID NO: 10 - protein, 628 aa, Bos taurus
MEPPDARAGARRAPRLLVLALLLAAPPGSKAEVRLSVPPLVEVMRGESVTLDCSPLGTHDYFMLEWFLVDRSGARHRLASAELRGSELRDKELNSRGRSPPYQLDSQGRLVLPEAQVGDERDYVCVVKAGAAGTAEATARLKVFAKPEAPEVSPNKGILSVMDDFAQEIATCSSRNGNPAPQIMWYRNGQPLAVPLEVNSEGYMTTRTVREASGLLSLTSTLYLRLHKPDREASFHCSVHYYLPAGQHGRLDGPSFSLTLHYPTEHVLFWLGSQSTAEGWVREGDSVQLLCQGDGSPTPEYTFFWLQDKQEDVLKTSLEGNLTLERVQRNQSGTYGCRVEDFDVPEDAELSKTLELRVAYLDSLELSAGEELSLPLHNSTTVTCSARGLPTPTLYWTKDSAPMGEDPTLSLHSVTFDSAGTYTCEAYMPRIPLLSRTRSFRLLVQGTPELKAKETQPKAEGSWTEGDEVTLICYARGYPKPKLTWSQLGGSPTEPAPGGQGWVSSSLTLKVTSALSQDGVSCEASNPLGNTHHVFHFGTVAPQTSQAGVAVMAVAISVALLLLVVAVFYCMRRKGRPGCCQWGEKGSPPPGEPKLSHSGSQRPEQTGLLMGSASGGAKHGSGGFGDEC

SEQ ID NO: 11 - protein, 507 aa, Artificial
  Human Lu gp-derived mutant Lu-del-5
EVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELNSQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 12 - protein, 418 aa, Artificial
  Human Lu gp-derived mutant Lu-del-4,5
EVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLNSQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 13 - protein, 324 aa, Artificial
  Human Lu gp-derived mutant Lu-del-3,4,5
EVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVNSQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 14 - protein, 622 aa, Mus musculus
MEPPDARAGLLWLTFLLSGYSGAQAELHVSVPPRVEVMRGEQVALDCTPREHPEHYVLEWFLVDGTGARHRLASVEPQGSEFLGTVHSLGRVPPYEVDSRGRLVIAKVQVGDGRDYVCVVKAGAAGTSEATSSVRVFATPEDTEVSPNKGTLSVMDQFAQEIATCSSNNGNPVPRITWYRNGQRLEVPMEVNQKGYITIRTVREASGLYSLTSTLYLRLHKDDRDADFHCAAHYDLPSGQHGRLDSHTFRLTLHYPTEHVEFWVGSPSTTEGWVREGDAVQLLCQGDGSPSPEYSFFRQQGTQEEQLNVNLKGNLTLEGVHRNQSGIYGCRVEDYDADEEVQLVKKLKLHVAYLDPLELSAPEELFVFLNSSSTVVNCSARGLPTPTVRWTKDSVTLADGPMLSLQSVTFDSAGTYTCEASTPTVPLLSRTQSFQLIVQGAPELKPNEIMPKSGNSWTEGDEVMLTCSARGFPEPKLTWSQRGDTPAEPPFEGRGWKSSSLMVKVTSALSREGVSCEASNIHGKKGHVFHFGSVAPQTAQAGVAVMAVAVSVGLLLLVVAAFYCMRRKGRPGCCRRAEKGAPPAREPELSHSGSERPEHTGLLMGGPSGGGRGGSGGFGDEC

SEQ ID NO: 15 - protein, 515 aa, Artificial
  Human Lu gp-derived mutant LUDelta1
MEPPDAPAQARGAPRLLLLAVLLAAHPDAQAEKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 16 - protein, 521 aa, Artificial
  Human Lu gp-derived mutant LuDelta2
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 17 - protein, 547 aa, Artificial
  Human Lu gp-derived mutant LUDelta4
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 18 - protein, 538 aa, Artificial
  Human Lu gp-derived mutant LuDelta5
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 19 - DNA, 24 bp, Artificial
  Synthetic oligonucleotide
gcccaggcggagtatcccacggag

SEQ ID NO: 20 - DNA, 24 bp, Artificial
  Synthetic oligonucleotide
ctccgtgggatactccgcctgggc

SEQ ID NO: 21 - DNA, 33 bp, Artificial
  Synthetic oligonucleotide
acttgggatccacttacctgtcagctccagcgt

SEQ ID NO: 22 - DNA, 33 bp, Artificial
  Synthetic oligonucleotide
acttgggatccacttacctgttggcgagccttg

SEQ ID NO: 23 - DNA, 33 bp, Artificial
  Synthetic oligonucletide
acttgggatccacttacctgtgtgctccgtggg

SEQ ID NO: 24 - protein, 8 aa, Artificial
  Human Lu gp mutation site sequence
SVMEDSAQ

SEQ ID NO: 25 - protein, 8 aa, Artificial
  Murine Lu gp mutation site sequence
SVMDQFAQ

SEQ ID NO: 26 - protein, 8 aa, Artificial
  Human Lu gp mutation site sequence
RTVREASG

SEQ ID NO: 27 - protein, 8 aa, Artificial
  Murine Lu gp mutation site sequence
RTVREASG

SEQ ID NO: 28 - protein, 8 aa, Artificial
  Human Lu gp mutation site sequence
LRKDDRDA

SEQ ID NO: 29 - protein, 8 aa, Artificial
  Murine Lu gp mutation site sequence
LHKDDRDA

SEQ ID NO: 30 - protein, 8 aa, Artificial
  Human Lu gp mutation site sequence
YPTEHVQF

SEQ ID NO: 31 - protein, 8 aa, Artificial
  Murine Lu gp mutation site sequence
YPTEHVEF

SEQ ID NO: 32 - protein, 8 aa, Artificial
  Human Lu gp mutation site sequence
PSPEYTLF

SEQ ID NO: 33 - protein, 8 aa, Artificial
  Murine Lu gp mutation site sequence
PSPEYSFF

SEQ ID NO: 34 - protein, 8 aa, Artificial
  Human Lu gp mutation site sequence
DEQEEVLN

SEQ ID NO: 35 - protein, 8 aa, Artificial
  Murine Lu gp mutation site sequence
GTQEEQLN

SEQ ID NO: 36 - protein, 8 aa, Artificial
  Human Lu gp mutation site sequence
EDYDAADD

SEQ ID NO: 37 - protein, 8 aa, Artificial
  Murine Lu gp mutation site sequence
EDYDADEE

SEQ ID NO: 38 - protein, 16 aa, Artificial
  Lu gp mutant construct end
LELRVAYLNSPQTSQA

(19) United States
(12) Patent Application Publication
(10) Pub. No.: US 2010/0211325 A1
(43) Pub. Date: Aug. 19, 2010

(54) LAMININ BINDING SITE

(76) Inventors: David John Anstee, Bristol (GB); Tosti John Mankelow, Bristol (GB); Stephen Frederick Parsons, Bristol (GB); Frances Adrienne Spring, Bristol (GB)

Correspondence Address: Kaser, Matthew R. Bell, Adam W.;Bell & Associates, 58 West Portal Avenue #121, San Francisco, CA, 94127, US

(21) Appl. No.: 12/312,104

(22) PCT Filed: Oct. 26, 2007
(86) PCT No.: PCT/GB2007/004092

§ 371 Date: Apr. 8, 2010

(30) Foreign Application Priority Data
Oct. 26, 2006 (GB) 0621279.9

(51) Int. Cl. G06F 19/00; C07K 14/00; C07H 21/04; G01N 33/68

(52) U.S. Cl. 702/19; 530/350; 536/23.1; 436/86

(57) ABSTRACT

The present invention in one aspect related to a ligand binding site for laminin, particularly those laminins comprising an α5 chain. The invention also encompasses binding sites on Lutheran glycoprotein (“Lu gp”) for binding laminin (“LM”) isoforms containing an α5 chain, mutant Lu gp molecules impaired at this binding site, antagonists and enhancers of the site, methods for identifying antagonists and enhancers, and uses of these molecules.

The present application relates to the Lutheran glycoprotein (“Lu gp”). In particular, the invention relates to binding sites on Lu gp such as the binding site for laminin (“LM”) isoforms containing an α5 chain, mutant Lu gp molecules impaired at this binding site, antagonists and enhancers of the site, methods for identifying antagonists and enhancers, and uses of these molecules.

Laminins are major adhesive and structural constituents of the extracellular matrix. They control numerous cellular activities such as adhesion, migration, proliferation, differentiation and apoptosis. Laminins are involved in the development of peripheral nerves, pancreas, placenta and digits and also play a role in muscle contraction and skin and hair growth. There are at least 16 types of laminin depending upon which combination of three subunits, α, β and γ, present in the heterotrimeric protein. Earlier studies on laminin adhesion have used laminins prepared from mouse Engelbreth Holm Schwarm (EHS) tumour cell cultures or from human placenta. Mouse EHS-laminin is the prototype laminin 111 (α1β1γ1). In contrast, crude human placental laminin preparations (merosin) contain mainly laminin 211 (α2β1γ1), laminin 221 (α2β2γ1), and a comparatively small proportion of laminin 511 (α5β1γ1) and laminin 521 (α5β2γ1).

The laminin α subunit contains a large carboxyl-terminal globular domain consisting of five laminin-type G (LG) modules, LG1 to LG5. LG modules are important in cell surface receptor-matrix interactions and the binding sites of at least six integrins, α-dystroglycan, heparin, and HNK-1/L2 have been mapped to them. Lysine and arginine residues in the C-terminal region of α1LG4, in positions 2766-2770, 2791-2793 and 2819-2820 together with R2869, K2858 and R2860 comprise the α-dystroglycan binding site. In α2LG5, K3027, K3030, K3088 and K3091 are important for heparin binding. Laminins 511 and 521 (also referred to herein as “LM511/521”) containing the α5 subunit also bind Lutheran glycoprotein (Lu gp) in the α5LG1-3 region (see Parsons et al., 2001, Blood 97, 312-320).

Previously, laminins have been numbered with Arabic numerals according to the order in which they were discovered, but as suggested by Aumailley et al. (2005; Matrix Biology 24: 326-332), a revised and simplified laminin nomenclature where the laminin is numbered using three Arabic numerals based on the α, β and γ chain numbers present will be used herein. This revised nomenclature is summarised in Table 1 of Aumailley et al. (2005) which shows, for example, that the laminins previously numbered 1, 2, 4, 10 and 11 are now abbreviated as laminins 111, 211, 221, 511 and 521, respectively.

The Lu gp, a member of the immunoglobulin superfamily (IgSF), consists of five IgSF domains and is expressed as two isoforms, of 85 and 78 kDa respectively (Parsons et al., 1987, Transfusion 27, 61-63; Parsons et al., 1995, Proc. Natl. Acad. Sci. USA 92, 5496-5500). The 78 kDa isoform (also known as BCAM or Lu[v13]) results from alternative splicing and lacks 40 C-terminal amino-acids within the cytoplasmic domain which contain an SH3 binding motif, a dileucine motif responsible for basolateral targeting (El Nemer et al., 1999, J. Biol. Chem. 274, 31903-31908) and five potential phosphorylation sites.

Lu gp is widely expressed in human tissue and carries antigens of the Lutheran blood group system on mature erythrocytes. Lu gp is expressed during late erythropoiesis at the orthochromatic erythroblast stage and it has been suggested that it functions either in cell extra-cellular matrix interactions during erythropoiesis or the trafficking of erythrocytes across the bone marrow sinus. Lu gp is thought to play a role in the pathophysiology of sickle cell disease by mediating adhesion of sickle cells to inflamed or damaged vascular endothelium. Recent studies have shown that higher than normal intracellular levels of cAMP in sickle erythrocytes lead to a PKA-mediated or Rap1-mediated signalling pathway resulting in increased adhesion of sickle cells to LM511/521. Furthermore, phosphorylation of the 85 kDa Lu gp at Serine 621 in adrenaline-stimulated K562 cells effects adhesion to LM511/521.

There has been disagreement in the scientific literature about which domains of Lu gp are involved in laminin 511/521 binding. Zen et al. (1999; J. Biol. Chem. 274: 728-734) reported that the fifth IgSF domain of Lu gp is critical for laminin 511/521 binding, whereas Parsons et al. (2001) deduced from their experiments that laminin 511/521 binding was effected through a binding site somewhere on IgSF domains 1-3 of Lu gp. El Nemer et al. (2001) corroborated the results of Parsons et al. (2001). The question was raised however as to whether there may be two laminin 511/521-binding regions on Lu gp.

The present inventors have resolved this issue as outlined herein, and their invention provides a powerful tool for identifying antagonists and enhancers of the Lu gp binding site.

According to a first aspect of the present invention, there is provided a ligand binding site for binding a laminin, in which the binding site is defined by the peptide sequence EDX₁D (SEQ ID NO: 1), where X₁ is any amino acid.

The binding site may further be defined by the peptide sequence EDX₁DAAX₂X₃ (SEQ ID NO: 2), where X₂ and X₃ are any amino acid.

X₁ may for example be selected from the group consisting of tyrosine (Y), glutamic acid (E) and aspartic acid (D).

X₂ and/or X₃ may each independently be selected from the group consisting of glutamic acid (E) and aspartic acid (D).

The binding site may for example have the sequence EDYDAADD (SEQ ID NO: 3) or EDYDADEE (SEQ ID NO: 4).

The laminin may be an isoform comprising an α5 chain (for example, laminins 511, 521, 511 or 521, such as laminins 511 or 521).

The ligand may be Lutheran glycoprotein (“Lu gp”) or a fragment thereof, with amino acid X₁ of the binding site corresponding to residue 311 of mature human Lu gp. This binding site may further comprise a hinge region between domains 2 and 3 of Lu gp, wherein the hinge region is defined by residues H230-F238 (SEQ ID NO: 5) of mature human Lu gp. The binding site may be defined further by one or two or more of the residues E132, D133, E180, D198, D199, E234, D269 and/or D316 of mature human Lu gp.

The human Lu gp sequence is available as Swissprot Accession No. P50895 (SEQ ID NO: 6). The full-length Lu gp protein is 628 residues in length, whereas the mature protein lacks an N-terminal signal domain of 31 amino acid residues. The amino acid sequence of mature human Lu gp (corresponding to residues 32-628 of P50895) is:

```
(SEQ ID NO: 7)
    1 EVRLSVPPLV EVMRGKSVIL DCTPTGTHDH YMLEWFLTDR SGARPRLASA EMQGSELQVT
  61 MHDTRGRSPP YQLDSQGRLV LAEAQVGDER DYVCVVRAGA AGTAEATARL NVFAKPEATE
121 VSPNKGTLSV MEDSAQEIAT CNSRNGNPAP KITWYRNGQR LEVPVEMNPE GYMTSRTVRE
181 ASGLLSLTST LYLRLRKDDR DASFHCAAHY SLPEGRHGRL DSPTFHLTLH YPTEHVQFWV
241 GSPSTPAGWV REGDTVQLLC RGDGSPSPEY TLFRLQDEQE EVLNVNLEGN LTLEGVTRGQ
301 SGTYGCRVED YDAADDVQLS KTLELRVAYL DPLELSEGKV LSLPLNSSAV VNCSVHGLPT
361 PALRWTKDST PLGDGPMLSL SSITFDSNGT YVCEASLPTV PVLSRTQNFT LLVQGSPELK
421 TAEIEPKADG SWREGDEVTL ICSARGHPDP KLSWSQLGGS PAEPIPGRQG WVSSSLTLKV
481 TSALSRDGIS CEASNPHGNK RHVFHFGTVS PQTSQAGVAV MAVAVSVGLL LLVVAVFYCV
541 RRKGGPCCRQ RREKGAPPPG EPGLSHSGSE QPEQTGLLMG GASGGARGGS GGFGDEC.
```

Residues 309-312 of mature human Lu gp corresponding to the laminin 511/521 binding site are underlined.

Based on the full-length Lu gp sequence (i.e. including the N-terminal signal peptide of 31 residues), residues 32-142 represent an Immunoglobulin (Ig)-like V-type domain 1, residues 147-257 represent an Ig-like V-type domain 2, residues 274-355 represent an I-type C2-type domain 3, residues 363-441 represent an I-type C2-type domain 4, and residues 448-541 represent an I-type C2-type domain 5. The binding site of the present invention is thus located in vivo on I-type C2-type domain 3 of human Lu gp.

The 85 kDa isoform of Lu gp is the major species found on red cells and the only isoform found in certain other tissues such as foetal liver and placenta (Parsons et al., 1995). The 78 kDa isoform of Lu gp shares identical sequence of the extracellular domains which bear the identified binding site. As both the 85 kDa and 78 kDa isoforms of Lu gp bind Laminin 511/521 (El Nemer et al., 1998, J. Cell Biol. 273: 16686-16693), the present invention encompasses a ligand which is either the 85 kDa or the 78 kDa Lu gp isoform or a fragment or variant of these.

The corresponding mouse conceptual protein for Lu gp has been deposited as GenBank Accession No. AAF14226 (SEQ ID NO: 8), the rat conceptual protein for Lu gp has been deposited as GenBank Accession No. AAH72479 (SEQ ID NO: 9), while the bovine Lu gp conceptual protein for Lu gp has been deposited as GenBank Accession No. AAF81749 (SEQ ID NO: 10).

The ligand binding site as defined herein on Lu gp polypeptides related to these sequences is within the scope of the present invention. The binding site of a polypeptide with Lu gp laminin binding activity as defined herein and at least 95%, 90%, 85%, 80%, 75%, 70%, 65%, 60%, 55%, 50%, 45% or 40% sequence identity, for example 40% sequence identity, with human Lu gp is included in the present invention.

The present binding site may thus be defined by corresponding residues of mature human Lu gp in non-human forms of Lu gp. Corresponding residues may be determined by alignment of non-forms of Lu gp with mature human Lu gp using alignment programs as defined below. Examples of corresponding residues are provided in Table 1 below. The mature Bos taurus Lu gp protein excludes an N-terminal signal sequence of 31 amino acid residues, the mature Rattus norvegicus Lu gp protein excludes an N-terminal signal sequence of 25 amino acid residues, and the mature Mus musculus Lu gp protein excludes an N-terminal signal sequence of 26 amino acid residues.

TABLE 1
Corresponding residues in mammalian Lu gp proteins.

| Mature | Mature Bos | Mature Rattus | Mature Mus |
|---|---|---|---|
| human | taurus | norvegicus | musculus |
| Lu gp | Lu gp | Lu gp | Lu gp |
| E132 | D132 | E132 | D130 |
| D133 | D133 | Q133 | Q131 |
| E180 | E180 | E180 | E178 |
| D198 | P198 | E198 | D196 |
| D199 | D199 | D199 | D197 |
| E234 | E234 | E234 | E232 |
| E269 | E269 | E269 | E267 |
| E309 | E309 | E309 | E307 |
| D310 | D310 | D310 | D308 |
| Y311 | F311 | Y311 | Y309 |
| D312 | D312 | D312 | D310 |
| D316 | D316 | E316 | E314 |
| H230-F238 | H230-F238 | H230-F238 | D228-F236 |

The binding site herein defined allows the identification of antagonists of the binding site, as elaborated below.

According to a further aspect of the invention, there is provided an isolated optionally recombinant polypeptide comprising the ligand binding site as defined herein, in which the polypeptide is a functional fragment of Lu gp or a homologue or variant of the functional fragment.

The isolated polypeptide may be up to 10, 15, 20, 25, 30, 35, 40, 45, 50, 100, 150, 200, 250, 300, 350, 400, 450, 500 or 550 amino acid residues in length.

The isolated polypeptide of the invention may exclude one or more or all of the deletion mutant polypeptides comprising the ligand binding site and as defined herein disclosed in Parsons et al. (1997 and 2001), Zen et al. (1999) and El Nemer et al. (1998 and 2001). These prior art deletion mutant polypeptides inadvertently included the binding site of the present invention. The presence of the binding site of the present invention was not realised or deduced from these prior art documents.

In particular, the excluded Parsons et al. (1997) polypeptides may be one or more of the human-derived Lu gp mutants defined as follows:

(SEQ ID NO: 11)

(1) Lu-del-5 (amino acid sequence-EVRLSVPPLV
EVMRGKSVIL DCTPTGTHDH YMLEWFLTDR SGARPRLASA
EMQGSELQVT MHDTRGRSPP YQLDSQGRLV LAEAQVGDER
DYVCVVRAGA AGTAEATARL NVFAKPEATE VSPNKGTLSV
MEDSAQEIAT CNSRNGNPAP KITWYRNGQR LEVPVEMNPE
GYMTSRTVRE ASGLLSLTST LYLRLRKDDR DASFHCAAHY
SLPEGRHGRL DSPTFHLTLH YPTEHVQFWV GSPSTPAGWV
REGDTVQLLC RGDGSPSPEY TLFRLQDEQE EVLNVNLEGN
LTLEGVTRGQ SGTYGCRVED YDAADDVQLS KTLELRVAYL
DPLELSEGKV LSLPLNSSAV VNCSVHGLPT PALRWTKDST
PLGDGPMLSL SSITFDSNGT YVCEASLPTV PVLSRTQNFT
LLVQGSPEL NS QTSQAGVAV MAVAVSVGLL LLVVAVFYCV
RRKGGPCCRQ RREKGAPPPG EPGLSHSGSE QPEQTGLLMG
GASGGARGGS GGFGDEC);

(SEQ ID NO: 12)

(2) Lu-del-4, 5 (amino acid sequence-EVRLSVPPLV
EVMRGKSVIL DCTPTGTHDH YMLEWFLTDR SGARPRLASA
EMQGSELQVT MHDTRGRSPP YQLDSQGRLV LAEAQVGDER
DYVCVVRAGA AGTAEATARL NVFARPEATE VSPNKGTLSV
MEDSAQEIAT CNSRNGNPAP KITWYRNGQR LEVPVEMNPE
GYMTSRTVRE ASGLLSLTST LYLRLRKDDR DASFHCAAHY
SLPEGRHGRL DSPTFHLTLH YPTEHVQFWV GSPSTPAGWV
REGDTVQLLC RGDGSPSPEY TLFRLQDEQE EVLNVNLEGN
LTLEGVTRGQ SGTYGCRVED YDAADDVQLS KTLELRVAYL NS
QTSQAGVAV MAVAVSVGLL LLVVAVFYCV RRKGGPCCRQ
RREKGAPPPG EPGLSHSGSE QPEQTGLLMG GASGGARGGS
GGFGDEG);
and

(SEQ ID NO: 13)

(3) Lu-del-3, 4, 5 (amino acid sequence-EVRLSVPPLV
EVMRGKSVIL DCTPTGTHDH YMLEWFLTDR SGARPRLASA
EMQGSELQVT MHDTRGRSPP YQLDSQGRLV LAEAQVGDER
DYVCVVRAGA AGTAEATARL NVFAKPEATE VSPNKGTLSV
MEDSAQEIAT CNSRNGNPAP KITWYRNGQR LEVPVEMNPE
GYMTSRTVRE ASGLLSLTST LYLRLRKDDR DASFHCAAHY
SLPEGRHGRL DSPTFHLTLH YPTEHV NS QTSQAGVAV
MAVAVSVGLL LLVVAVFYCV RRKGGPCCRQ RREKGAPPPG
EPGLSHSGSE QPEQTGLLMG GASGGARGGS GGFGDEC).

The excluded Parsons et al. (2001) polypeptides may be one or more of those designated Del 5, Del 4-5 and Del 3-5 (equivalent to Lu-del-5, Lu-del-4,5, and Lu-del-3,4,5 Lu gp mutants as defined above, respectively) linked with the Fc domains of human IgG (derived from the pIg vector; see Simmons, 1993, In: Cellular Interactions in Development: A Practical Approach, Hartley [ed.], IRL Press, Oxford UK, pp 93-127), the polypeptide containing the extracellular domains of mouse Lu gp (derived from GenBank Accession No. AF246667; SEQ ID NO: 14), and the polypeptide consisting of this mouse Lu gp polypeptide linked with the above-mentioned Fc domains of human IgG.

The excluded Zen et al. (1999) polypeptides may be one or more of the human-derived Lu gp mutants defined as follows:

(SEQ ID NO: 15)

(1) LUΔ1 (residues M1 to E32 + K146 to C628 of
full-length human Lu gp, having the amino acid
sequence-MEPPDAPAQA RGAPRLLLLA VLLAAHPDAQ AE KPEAT
EVSPNKGTLS VMEDSAQEIA TCNSRNGNPA PKITWYRNGQ
RLEVPVEMNP EGYMTSRTVR EASGLLSLTS TLYLRLRKDD
RDASFHCAAH YSLPEGRHGR LDSPTFHLTL HYPTEHVQFW
VGSPSTPAGW VREGDTVQLL CRGDGSPSPE YTLFRLQDEQ
EEVLNVNLEG NLTLEGVTRG QSGTYGCRVE DYDAADDVQL
SKTLELRVAY LDPLELSEGK VLSLPLNSSA VVNCSVHGLP
TPALRWTKDS TPLGDGPMLS LSSITFDSNG TYVCEASLPT
VPVLSRTQNF TLLVQGSPEL KTAEIEPKAD GSWREGDEVT
LICSARGHPD PKLSWSQLGG SPAEPIPGRQ GWVSSSLTLK
VTSALSRDGI SCEASNPHGN KRHVFHFGTV SPQTSQAGVA
VMAVAVSVGL LLLVVAVFYC VRRKGGPCCR QRREKGAPPP
GEPGLSHSGS EQPEQTGLLM GGASGGARGG SGGFGDEC);

(SEQ ID NO: 16)

(2) LUΔ2 (residues M1 to T150 + L258 to C628 of
full-length human Lu gp, having the amino acid
sequence-MEPPDAPAQA RGAPRLLLLA VLLAAHPDAQ
AEVRLSVPPL VEVMRGKSVI LDCTPTGTHD HYMLEWFLTD
RSGARPRLAS AEMQGSELQV TMHDTRGRSP PYQLDSQGRL
VLAEAQVGDE RDYVCVVRAG AAGTAEATAR LNVFAKPEAT LTL
HYPTEHVQFW VGSPSTPAGW VREGDTVQLL CRGDGSPSPE
YTLFRLQDEQ EEVLNVNLEG NLTLEGVTRG QSGTYGCRVE
DYDAADDVQL SKTLELRVAY LDPLELSEGK VLSLPLNSSA
VVNCSVHGLP TPALRWTKDS TPLGDGPMLS LSSITFDSNG
TYVCEASLPT VPVLSRTQNF TLLVQGSPEL KTAEIEPKAD
GSWREGDEVT LIGSARGHPD PKLSWSQLGG SPAEPIPGRQ
GWVSSSLTLK VTSALSRDGI SCEASNPHGN KRHVFHFGTV
SPQTSQAGVA VMAVAVSVGL LLLVVAVFYC VRRKGGPCCR
QRREKGAPPP GEPGLSHSGS EQPEQTGLLM GGASGGARGG
SGGFGDEC);

(SEQ ID NO: 17)

(3) LUΔ4 (residues M1 to P363 + Q358 to C628 of
full length human Lu gp, having the amino acid
sequence-MEPPDAPAQA RGAPRLLLLA VLLAAHPDAQ
AEVRLSVPPL VEVMRGKSVI LDCTPTGTHD HYMLEWFLTD
RSGARPRLAS AEMQGSELQV TMHDTRGRSP PYQLDSQGRL
VLAEAQVGDE RDYVCVVRAG AAGTAEATAR LNVFAKPEAT
EVSPNKGTLS VMEDSAQEIA TGNSRNGNPA PKITWYRNGQ
RLEVPVEMNP EGYMTSRTVR EASGLLSLTS TLYLRLRKDD
RDASFHCAAH YSLPEGRHGR LDSPTFHLTL HYPTEHVQFW
VGSPSTPAGW VREGDTVQLL CRGDGSPSPE YTLFRLQDEQ
EEVLNVNLEG NLTLEGVTRG QSGTYGCRVE DYDAADDVQL
SKTLELRVAY LDP QGSPEL KTAEIEPKAD GSWREGDEVT
LICSARGHPD PKLSWSQLGG SPAEPIPGRQ GWVSSSLTLK
VTSALSRDGI SCEASNPHGN KRHVFHFGTV SPQTSQAGVA
VNAVAVSVGL LLLVVAVFYC VRRKGGPCCR QRREKGAPPP
GEPGLSHSGS EQPEQTGLLM GGASGGARGG SGGFGDEC);
and

(SEQ ID NO: 18)

(4) LUΔ5 (residues M1 to T452 + Q543 to C628 of
full length human Lu gp, having the amino acid
sequence-MEPPDAPAQA RGAPRLLLLA VLLAAHPDAQ
AEVRLSVPPL VEVMRGKSVI LDCTPTGTHD HYMLEWFLTD
RSGARPRLAS AEMQGSELQV TMHDTRGRSP PYQLDSQGRL
VLAEAQVGDE RDYVCVVRAG AAGTAEATAR LNVFAKPEAT
EVSPNKGTLS VMEDSAQETA TCNSRNGNPA PKITWYRNGQ
RLEVPVEMNP EGYMTSRTVR EASGLLSLTS TLYLRLRKDD
RDASFHCAAH YSLPEGRHGR LDSPTFHLTL HYPTEHVQFW
VGSPSTPAGW VREGDTVQLL CRGDGSPSPE YTLFRLQDEQ
EEVLNVNLEG NLTLEGVTRG QSGTYGCRVE DYDAADDVQL
SKTLELRVAY LDPLELSEGK VLSLPLNSSA VVNCSVHGLP
TPALRWTKDS TPLGDGPMLS LSSITFDSNG TYVCEASLPT
VPVLSRTQNF TLLVQGSPEL KT QTSQAGVA VMAVAVSVGL
LLLVVAVFYC VRRKGGPCCR QRREKGAPPP GEPGLSHSGS
EQPEQTGLLM GGASGGARGG SGGFGDEC).

The excluded El Nemer et al. (1998) polypeptide may be the human-derived Lu gp mutant consisting of the extracellular domains of Lu gp linked with the Fc domains of human IgG (derived from the pIg vector; see above).

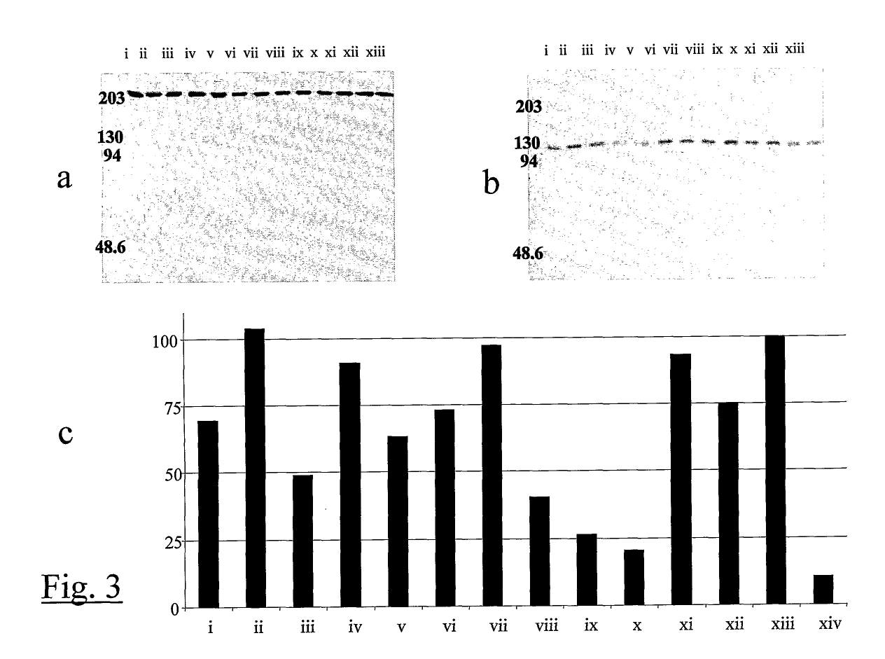

The El Nemer et al. (2001) polypeptides may be one or more of the human-derived Lu gp mutants comprising various domains of Lu gp linked with the Fc domains of human IgG (derived from the pIg vector) and were designated: Lu12345-Fc (equivalent to the excluded polypeptide from El Nemer et al., 1998); Lu1234-Fc; Lu123-Fc; Lu13-Fc; Lu23-Fc; Lu3-Fc; LuΔPSPEY-Fc; LuΔLNVNL-Fc; Lu N321A-Fc; and Lu R292A-Fc (see FIG. 3 of El Nemer et al., 2001).

It is noted that the naturally occurring SNPs within Lu gp, as characterised in Crew et al. (2003, Transfusion 43: 1729-1737), are outside the scope of this aspect of the invention.

In an embodiment of the present invention, the isolated polypeptide comprising the ligand binding site as defined herein excludes an Fc domain such as one or more of the Fc domains of human IgG.

In a further aspect of the invention, there is provided a method for identifying (i) a molecule or substance which is an antagonist, or (ii) a molecule or substance which is an enhancer, of a Lu gp binding site for a laminin isoform having an α5 chain (for example, laminins 511, 521, 511 or 521, such as laminin 511 or laminin 521), in which the method comprises identifying, on the basis of biological activity and/or structural properties (for example, spatial arrangement of key chemical elements) and/or chemical properties and/or electronic environment of the binding site and/or the molecule or substance, a molecule or substance which replicates and/or interacts with the Lu gp laminin binding site as defined herein. The Lu gp binding site may further comprise an amino acid residue corresponding to H235 of human Lu gp.

The method of the invention makes it possible to identify molecules or substances that possess the desired activity with respect to the Lu gp binding site for a laminin isoform having an α5 chain (for example laminin 511 or laminin 521).

The structural and/or chemical properties and/or electrical environment of the molecule or substance may be defined by a pharmacophore. A pharmacophore is a set of structural features in a molecule or substance that is recognised at the binding site and is responsible for that molecule's biological activity. Pharmacophoric units (also known as “features” or “agons”) defining the pharmacophore may be determined by complementarity (i.e. replication and/or interaction) with the binding site on the basis of structural characteristics (for example, spatial arrangement of key residues) and/or chemical characteristics and/or electronic environment of the binding site.

A molecular model for human Lu gp and binding site for use in the method is elaborated below.

In one aspect of the invention, there is provided a method of generating a pharmacophore model for an antagonist or enhancer of the ligand (for example, Lu gp) binding site as defined herein. The method comprises the steps of generating from the three-dimensional structure (or “conformer”) of the binding site a pharmacophore model for an antagonist or enhancer of the binding site, in which the pharmacophore model comprises three or more of the binding site residue features selected from the group consisting of a hydrogen bond donor feature, a hydrogen bond acceptor feature, a hydrophobic region feature, an ionisable region feature, and a ring aromatic feature, arranged in three-dimensional space.

The pharmacophore model may then be used to screen for molecules which replicate and/or interact with the Lu gp laminin binding site as defined herein.

The method steps may be performed in silico, using molecular modeling software. Using computational chemistry, pharmacophore and/or other requirements for the antagonist or enhancer molecule are determined on the basis of the binding site structure and/or chemical properties and/or electronic environment. Chemical and/or biological databases can then be searched for candidate antagonist molecules or substances with the correct pharmacophore and/or other determined requirements.

The use of computational quantitative structure activity relationship (QSAR) modeling techniques to identify antagonist or enhancer molecules or substances is therefore encompassed by the present invention.

Several methods and packages are known in the prior art for use in performing the method of obtaining a pharmacophore model, for example the UNITY system provided in the Tripos SYBYL molecular modeling suite of programs (Tripos, St Louis, Mo., US).

The model makes it possible to screen, discover and select molecules with the desired properties.

The method may comprise a further step of verifying experimentally (for example, in vitro or in vivo) whether and/or to what extent the identified molecule or substance is an antagonist or enhancer of the Lu gp laminin binding site. The experimental section below provides examples of tests which may be used to verify whether an identified putative antagonist is functionally effective.

In a further aspect of the invention, there is provided a method of manufacturing a molecule which is an antagonist or enhancer of a Lu gp binding site for a laminin isoform comprising an α5 chain (for example laminin 511 or laminin 521), comprising the steps of identifying the molecule according to the invention method as described herein and then manufacturing the molecule.

In another aspect of the invention there is provided a pharmaceutical composition comprising the molecule identified and/or manufactured by the invention methods described herein.

Also provided is the use of a molecule identified and/or manufactured by the invention methods described herein, or the pharmaceutical composition described above, in the treatment of a disease. For example, the disease may be cancer, sickle cell disease, deep vein thrombosis (DVT), malaria, heart disease, vascular complications, diabetes, β-thalassemia, or a thrombotic complication of haematological diseases. Lu gp is a known marker for cancer, and the use of an antagonist molecule which affects Lu gp-laminin 511/521 interaction for the treatment of cancer is within the scope of the present invention.

A molecule which enhances binding between the Lu gp laminin binding site and laminin 511/521 may, for example, be used as a clotting agent.

Another aspect of the invention provides a recombinant polypeptide having at least 95%, 90%, 85%, 80%, 75%, 70%, 65%, 60%, 55%, 50%, 45% or 40% identity, for example 40% sequence identity, to wild-type mature human Lu gp, in which an amino acid in the polypeptide corresponding to residue 312 in mature human Lu gp is other than aspartic acid, such that the polypeptide has impaired affinity for a laminin isoform comprising an α5 chain (for example, laminin 511 or laminin 521) compared with the corresponding wild-type Lu gp.

In this recombinant polypeptide, an amino acid in the polypeptide corresponding to residue 235 in mature human Lu gp may be other than histidine.

Additionally or alternatively, an amino acid in the polypeptide corresponding to residue 309 in mature human Lu gp may be other than glutamic acid and/or an amino acid corresponding to residue 310 in mature human Lu gp may be other than aspartic acid.

Furthermore, an amino acid in the polypeptide corresponding to residue 198, 199 and/or 269 in mature human Lu gp may be other than aspartic acid.

Also, an amino acid in the polypeptide corresponding to residue 133 in mature human Lu gp may be other than aspartic acid or glutamine.

An amino acid in the polypeptide corresponding to residue 133, 198, 199, 235, 269, 309, 310 and/or 312 in mature human Lu gp may be been subjected to point mutation and/or deletion.

The amino acid in the polypeptide corresponding to residue 133, 198, 199, 235, 269, 309, 310 and/or 312 in mature human Lu gp may be alanine.

In another aspect of the invention, there is provided an Lu gp polypeptide having impaired affinity for a laminin isoform comprising an α5 chain (for example, laminin 511 or laminin 521) compared with the corresponding wild-type Lu gp, in which one or more of the residues in the laminin binding site defined as defined herein has been mutated compared with wild-type Lu gp, provided that at least the amino acid corresponding to residue 312 in mature human Lu gp is other than aspartic acid.

Excluded from the scope of the polypeptide having impaired affinity for a laminin isoform comprising an α5 chain are one or more or all of the recombinant polypeptides impaired in the laminin binding site and as defined herein disclosed in Parsons et al. (1997 and 2001), Zen et al. (1999) and El Nemer et al. (2001). These prior art recombinant polypeptides may have inadvertently included mutations in or of the binding site of the present invention.

For example, the excluded Parsons et al. (1997) polypeptides may be one or more of those designated Lu-del-3,4,5 and Lu-del-2,3,4,5; the excluded Parsons et al. (2001) polypeptides may be one or more of those designated Del 3-5 and Del 2-5; the excluded Zen et al. (1999) polypeptides may be one or more of those designated LuΔ3 and Lu5; the excluded El Nemer et al. (2001) polypeptides may be one or more of those designated Lu12-Fc, Lul-Fc, Lu4-Fc, Lu5-Fc, Lu45-Fc, Lu12450Fc and LuΔRVEDY-Fc.

Further provided according to the present invention is a kit for testing binding of a laminin isoform comprising an α5 chain (for example, laminin 511 or laminin 521) with Lu gp or a fragment thereof, comprising an Lu gp polypeptide of the present invention.

There is also provided a molecular model for Lu gp as described herein with reference to the accompanying drawings.

Use of this molecular model in an in silico method for identifying a molecule which is an antagonist of a Lu gp binding site for a laminin isoform having an α5 chain (for example laminin 511 or laminin 521) is a further aspect of the invention.

Sequence identity or similarity between amino acid sequences can be determined by comparing an alignment of the sequences. When an equivalent position in the compared sequences is occupied by the same amino acid, then the molecules are identical at that position. When the equivalent site is occupied by a similar amino acid residue (for example, similar in steric and/or electronic nature), then the molecules can be referred to as similar (also known as homologous) at that position. Scoring an alignment as a percentage of identity is a function of the number of identical amino acids at positions shared by the compared sequences, while scoring an alignment as a percentage of similarity is a function of the number of identical and similar amino acids at positions shared by the compared sequences.

When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity and/or similarity involves the production of an optimal alignment, taking into consideration gap penalties.

Suitable computer programs for carrying out sequence comparisons are widely available in the commercial and public sector. Examples include the Gap program (Needleman & Wunsch, 1970, J. Mol. Biol. 48, 443-453) and the FASTA program (Altschul et al., 1990, J. Mol. Biol. 215, 403-410). Gap and FASTA are available as part of the Accelrys GCG Package Version 11.1 (Accelrys, Cambridge, UK), formerly known as the GCG Wisconsin Package. The FASTA program can alternatively be accessed publicly from the European Bioinformatics Institute (http://www.ebi.ac.uk/fasta) and the University of Virginia (http://fasta.biotech.virginia.edu/fasta_www/cgi). FASTA may be used to search a sequence database with a given sequence or to compare two given sequences (see http://fasta.bioch.virginia.edu/fasta_www/cgi/search_frm2.cgi). Typically, default parameters set by the computer programs should be used when comparing sequences. The default parameters may change depending on the type and length of sequences being compared. A sequence comparison using the FASTA program may use default parameters of Ktup=2, Scoring matrix=Blosum50, gap=−10 and ext=−2.

Also within the scope of the invention are variants and/or derivatives of the polypeptides of the invention. The terms “variant” or “derivative” includes any substitution of, variation of, modification of, replacement of, deletion of or addition of one or more amino acids from or to the polypeptide sequence providing the resultant amino acid sequence retains substantially the same activity as the unmodified sequence.

As used herein, the term “antagonist” means a molecule or substance which binds, inhibits or negatively interacts with the Lu gp binding site as defined herein, wherein the molecule or substance is other than a naturally-occurring laminin isoform with an α5 chain. The term “antagonist” may be considered as synonymous with the term “inhibitor”. An “enhancer” is a molecule or substance which causes increased binding between the Lu gp binding site and the laminin isoform with an α5 chain (such as laminin 511/521) which binds to the binding site.

Embodiments of the invention will be described hereafter with reference to the accompanying drawings, of which:

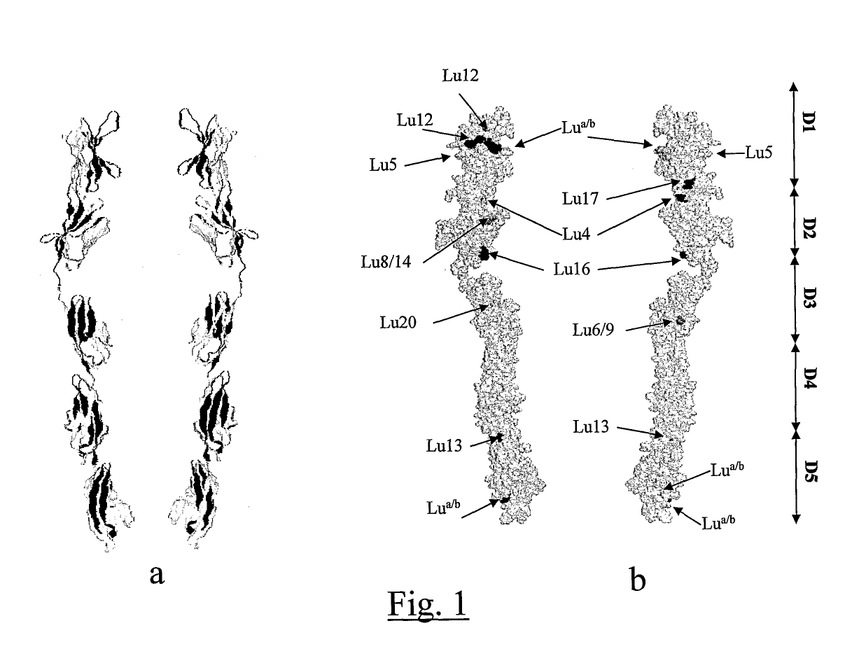

FIG. 1 shows a molecular model of the extracellular IgSF domains of Lu gp. Each domain of Lu gp was modelled on the crystal structure of suitable sub-set Ig domains with the highest sequence identity to the Lu gp domains. a) Lu gp is shown in two orientations rotated 180 degrees on the y axis. The ABE face of each domain is shown in grey and the CFG face shown in black. b) The location of the polymorphic amino-acid positions that define the antigens of the Lu blood group system are displayed on Lu gp in two orientations rotated 180 degrees on the y axis. The following blood groups are shown: Domain 1, Lua/b, Lu5, Lu12 and Lu17; Domain 2, Lu4, Lu8/14 and Lu16; Domain 3, Lu6/9 and Lu20; Domain 5, Aua/b and Lu13;

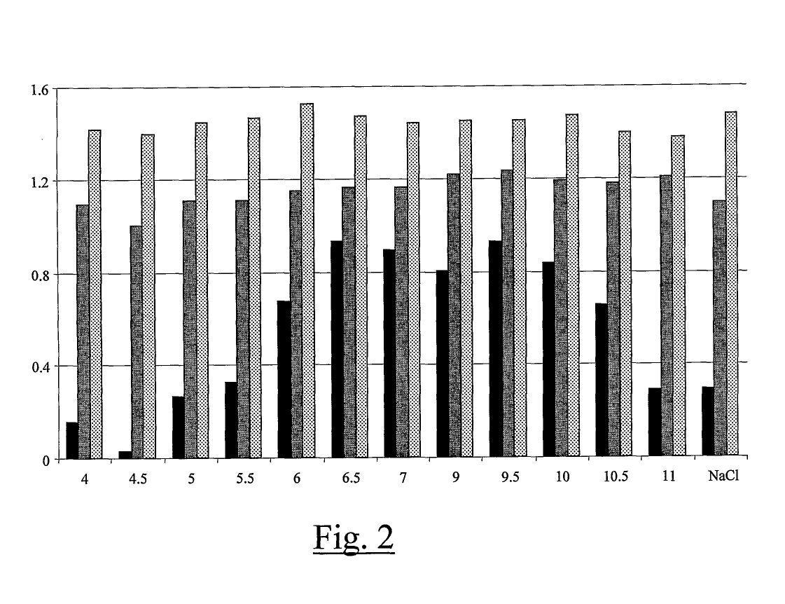

FIG. 2 is a histogram showing LM511/521 binding to Lu gpFc is affected by buffer conditions in an ELISA based assay system, with assay conditions shown on the X axis and OD₄₅₀ on the Y axis. The adherence of LM511/521 to Lu gpFc was tested in different 0.1M buffers from pH4.0 to pH11.0 (acetate buffers pH4.0 to pH 5.5, phosphate buffers pH 6.0 to pH 7.0 and glycine buffers pH 9.0 to pH 11.0) and at high salt (1M NaCl pH 7.4) in an ELISA system. Lu gp was either pre-exposed to PBS-BSA and LM511/521 added in the different buffers (black bars), pre-exposed to the different buffers and LM511/521 added in PBS-BSA (grey bars) or LM511/521 was coated directly to the plate and then incubated with the different buffers (speckled). The wells were then incubated with the rabbit anti-Laminin and swine anti Rabbit HRP antibodies as described in the materials and methods section. A negative control Fc fusion protein Muc-18 was also tested in parallel in each buffer (not shown). Background binding was in the range OD450 0.18 to 0.64 and was subtracted from the results obtained using Lu gpFc. Conditions were performed in duplicate on each ELISA plate and the results shown are representative of two separate assays;

FIG. 3 depicts Western blots and a histogram showing mutation of certain acidic residues of Lu gp reduces LM511/521 binding. Western blots using Bric 224 (a) followed by a rabbit anti-mouse HRP secondary Ab was performed on non-reduced samples (0.2 μgs) and using an anti human Fc HRP conjugate (b) on reduced samples. The level of adherence to LM511/521 of mutant Lu gp compared with the native protein was tested in an ELISA (c). The graph in (c) shows the protein on the X axis and the percentage of control at OD₄₅₀ on the Y axis. The Lu gpFc was captured onto a plate coated with an anti-Fc antibody, after the addition of LM511/521 detection was via a rabbit anti Laminin antibody and an anti rabbit HRP conjugated antibody. Lanes for a, b and c are i—E132A/D133A, ii—E180A, iii—D198A/D199A, iv E234A, v—D269A, vi—D280A, vii—D281A, viii—E309A, ix—E310A, x—D312A, xi—D315A, xii—D316A, xiii—Native Lu, and xiv—Muc18 (not a and b). On each ELISA plate the proteins were assayed in duplicate and the result shown are the mean of two separate ELISA plates. The results are expressed as the percentage of the absorption seen from 0.05 nM of the native protein (00450=0.95);

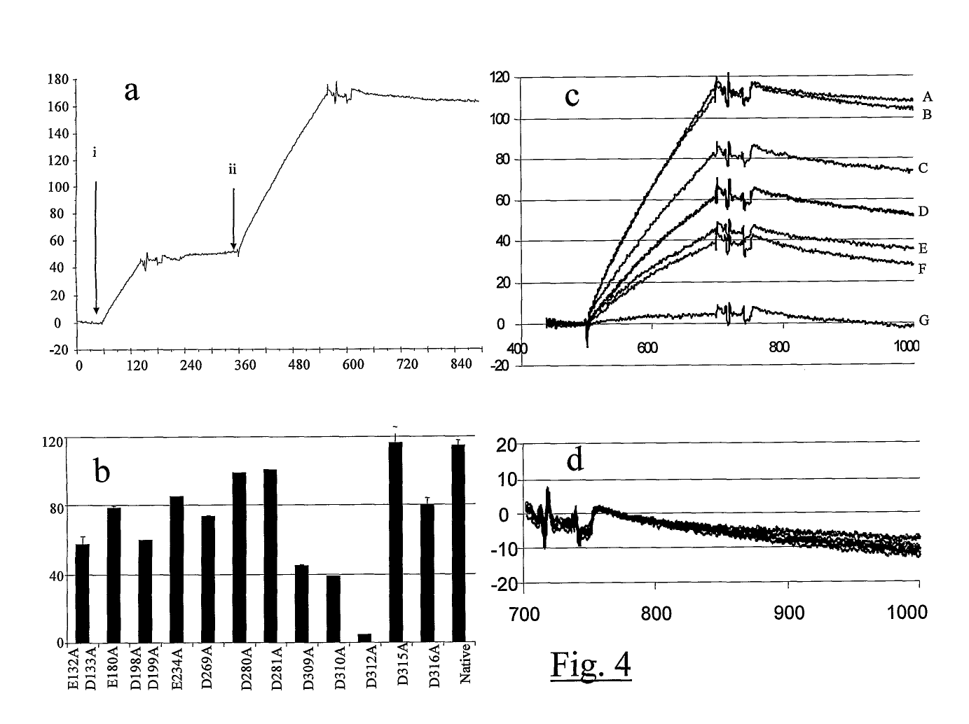

FIG. 4 depicts graphs showing the level of native and mutant Lu gpFc binding to LM511/521 in a Surface Plasmon Resonance (Biacore) assay system. The level of adherence to LM511/521 of mutant Lu gp compared with the native protein was tested by surface plasmon resonance in a BiacoreX. The Lu gpFc was captured onto CM5 chip coated with protein A to give a total RU shift of 40 units. The response to a 100 μl injection of 10 nM LM511/521 was then measured and each mutant protein was assayed twice. In a typical sensorgram of LM511/521 binding to native Lu gpFc (a) it is clearly seen when Lu gpFc (i) and LM511/521 (ii) are added onto the chip. In (a), the X axis shows time in s, while the Y axis shows response units (RU). The mean change in RU; Y axis over the course of a 100 μl injection of 10 nM LM511/521 of two Biacore assays per protein (X axis in (b). Combined together the sensorgrams of a representative sample of the different proteins show clear differences in the association rates (c), but very little difference in the disassociation rates (d). In (c), A=D315A, B=native, C=D316A, D=198A/D199A, E=E309A, F=D310A and G=D312A Lu gbFc proteins;

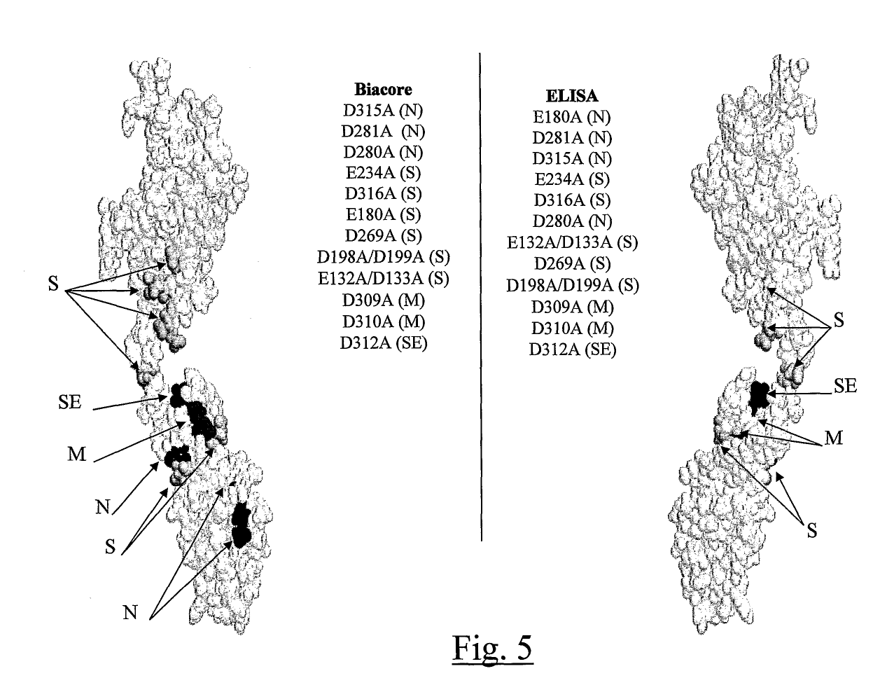

FIG. 5 is a model of Lu gp showing the charged amino-acid residues on the surface of Lu gp that are involved in adhesion to LM511/521. The model of Lu gp domains 2 and 3 is shown in two orientations and is white in colour. Indicated are the positions of acidic residues that have been mutated to alanine. The residues are coded according to their effect on LM511/521 binding, in which N=no effect, S=slight effect, M=marked effect and SE=severe effect. Biacore and ELISA rankings of the effect of the mutations (see Table 3) are also shown;

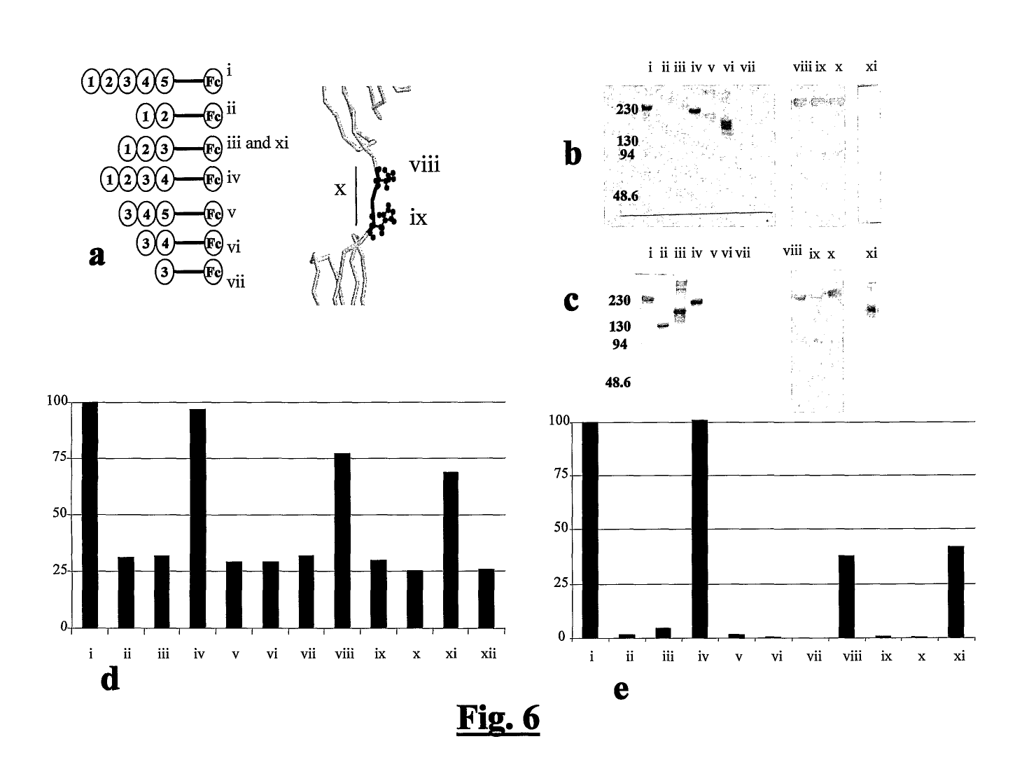

FIG. 6 shows data relating to LM511/521 adhesion to Lu gp domain deletions and mutations in the hinge region. Key—(a) native protein (i), D1+2 (ii), D1+2+3a (iii), D1+2+3+4 (iv), D3+4+5 (v), D3+4 (vi), D3 (vii) T233P (viii), H235P (ix), 0233-235 (x) and D1+2+3b (xi). Different Lu domain deletion proteins were made (ai-vii) along with two proteins containing changes to the domain 2 to 3 hinge T233P and H235P (aviii-ix) and Δ233-235 (x) darker shade). A western blot was performed on each protein using BRIC 221 (epitope on domain 4) (b), BRIC 224 (epitope on domain 1) and an anti Ig Fc (not shown) (c). Protein standards are 230, 130, 94 and 48.6 kDa. The level of adherence to LM511/521 of these proteins was tested in two separate assay systems. In the ELISA assay (d) adhesion to LM511/521, observed as an average OD450, of two ELISA's in a 96 well plate coated with 0.05 nM Lu protein and shown as the % binding to the native Lu (OD450=0.99). This was compared with the level of LM511/521 binding observed by an IgSF Fc control Muc18 (d xii). In the Biacore assay (e) adhesion of the Lu proteins to LM511/521 is shown as a % of the total change in RU's during a 100 μl injection of 10 nM LM511/521 at a flow rate of 30 μl/min observed by the native Lu gpFc protein (129 RU's). In (d) and (e), the X axis indicates the test protein and the Y axis shows percentage of control adhesion;

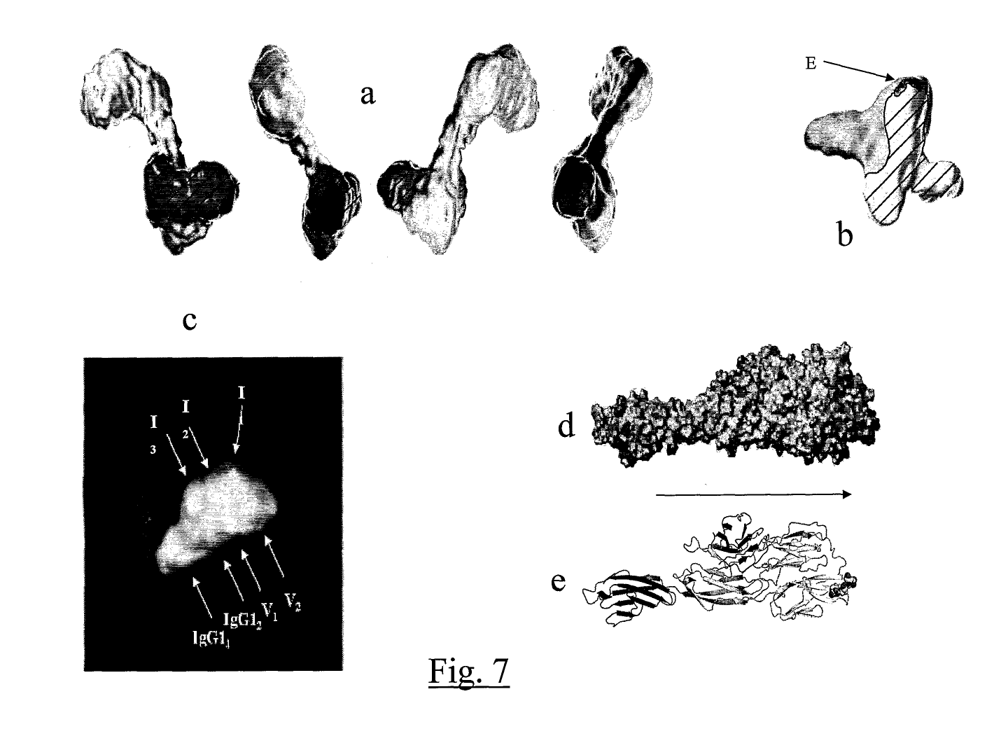

FIG. 7 shows protein Tomography™ analysis of monomeric Lu gpFc and monomeric Lu gpFc bound to LM511/521. A sample containing Lu gpFc bound to LM511/521 (a), Bric 108 bound to Lu gpFc (b) and Lu gpFc (c) were analyses using Protein Tomography™. In (a), Lu gpFc (hatched) is shown adopting a bent confirmation when bound to LM511/521 (clear). Domain orientation of Lu gpFc (clear) was performed using the Bric 108 (hatched) antibody which binds an epitope (“E”) on domain 1 of Lu gp in (b). (c) shows the monomeric form of Lu gpFc is shown in a bent conformation with the suspected relative positions of each Ig domain labelled. The model of Lu gp was altered to a bent conformation using information gained from the PatchDock web server and is shown in (d) with an arrow indicating the general gradient of electrostatic potential on the molecular surface moving from positive to negative (the most negative electrostatic potential being found on the surface to the right of the structure), and as ribbon with E309, D310 and D312 in ball and stick (e); and

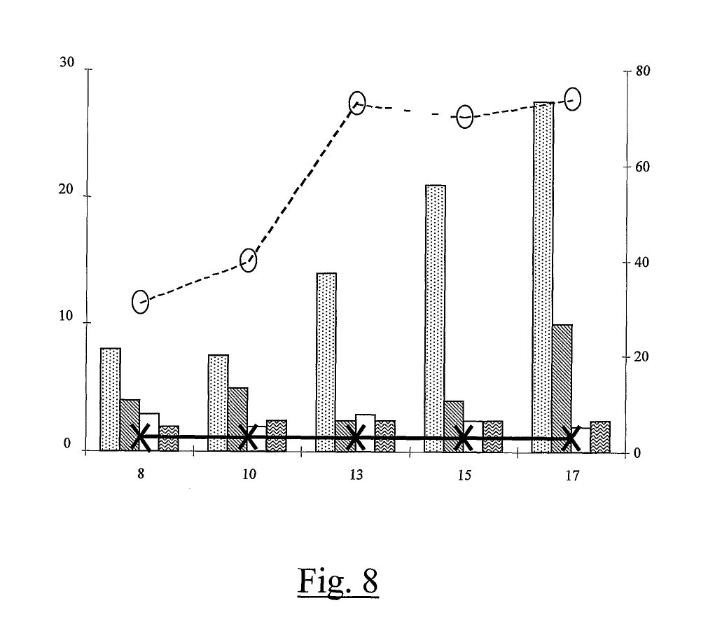

FIG. 8 is a graph showing the adhesion of normal and Lu gp negative erythroblasts to LM511/521 at different stages of maturation. The adherence of erythroblasts to LM511/521 (Invitrogen) was tested in a cell adhesion assay as described in the Materials and Methods on days 8, 10, 13, 15 and 17 (shown on X axis) of an erythroblastic culture. Adhesion of normal erythroblasts to LM511/521 (dotted), LM111 (diagonal line) and Lu gp negative erythroblasts to LM511/521 (no pattern) and LM111 (zig zag) are shown as the % number of erythrocytes adhering (the left Y axis). Expression of Lu gp was performed by fax analysis and is shown as the % Lu expression (the right Y axis): Normal culture (--o--) and In(Lu) culture (-X-). Binding of control LuK562 cells to LM511/521 was 70-85% and less than 10% to LM111 (results not shown).

### DETAILED DESCRIPTION OF THE INVENTION

### Example 1

### Introduction

The nature of Lu gp-laminin 511/521 interaction and its functional importance has been poorly understood. In order to solve the problem, we have constructed a molecular model of Lu gp and targeted surface-exposed clusters of acidic (negatively charged) amino-acids within domains 1, 2 and 3 for mutation to alanine. The mutant Lu gps (expressed as Fc fusion proteins) were tested for laminin 511/521 (“LM511/521”) binding and a region of negative charge on domain 3, proximal to an unusually extended and potentially flexible hinge region of 6-8 residues between domains 2 and 3, was identified as the LM511/521 binding site. Tomographical reconstruction of unbound Lu gp and Lu gp-LM511/521 complexes show Lu gp folded at this hinge region to expose the binding site on domain 3. Mutation and deletion of residues in the hinge region suggested that flexibility at the hinge region is essential for LM511/521 binding to Lu gp. These results identify Lu gp binding to LM511/521 as a novel type of protein:protein interaction.

### Materials and Methods

### Lu gp Domains 1-5 Homology Model

In order to produce an homology model of the extracellular region of Lu gp each of the five IgSF domains was assigned to a sub-set according to amino acid sequence. Domains 1 and 2 were classified as V-set, domains 3, 4 and 5 were classified as I-set. From the available structures of the appropriate sub-set the domains with the highest sequence identity to the Lu gp domains were selected as template structures. For domains 1, 2 and 3 the templates consisted of single domains, for domains 4 and 5 a continuous two domain template was available. The templates used were as follows; Lu gp domain 1 and 2 chain A of PDB 1fo0 (http://www.rcsb.org/pdb/navbarsearch.do?newSearch=yes&isAuthorSearch=no&radioset=All&inputQuickSearch=1fo0&image.x=23&image.y=³), Lu gp domain 3 chain b of PDB 1gl4 (http://www.rcsb.org/pdb/navbarsearch.do?newSearch=yes&isAuthorSearch=no&radioset=All&inputQuickSearch=1gl4&image.x=44&image.y=9) and chain A domains 1 and 2 of PDB 1cs6 for Lu gp domains 4 and 5 (http://www.rcsb.org/pdb/navbarsearch.do?newSearch=yes&isAuthorSearch=no&radioset=All&inputQuickSearch=1cs6&image.x=33&image.y=2).

Sequences were initially aligned using the ClustalW routine within Megalign 5.06 (DNASTAR, Madison, Wis., USA). Manual adjustments were made to ensure alignment of IgSF and sub-set signature residues and the location of inserts and deletions in loop regions. Consideration was also given to secondary structure predictions from the Jpred (Cuff et al., 1998, Bioinformatics. 14, 892-893; Cuff & Barton, 1999, Proteins 34, 508-519) and SAMT02 (Karplus et al., 2003, Proteins 53 Suppl 6, 491-496) servers. These sequence alignments and secondary structure predictions were used as input for Modeler (Marti-Renom et al., 2000, Ann. Rev. Biophys. Biomol. Struct. 29, 291-325) which generated the three-dimensional models.

In order to predict the inter-domain orientations multiple models were constructed by overlaying the Lu gp domains on experimental structures of two continuous immunoglobulin domains, then joining the domains and energy minimizing the linker region using SYBYL7.1 (Tripos, St Louis, Mo., USA). The orientation which resulted in the highest quality model, as assessed by PROCHECK (Laskowski et al., 1993, J. Appl. Cryst. 26, 283-291), was selected and used in the final five domain structure.

Preparation of Mutant and Native Lu gpFc Fusion Proteins

Point mutations were inserted into human Lutheran cDNA clones (Parsons et al., 1997, Blood 89, 4219-4225) encoding the five extracellular domains in pIg vector by PCR amplification as described previously (for ICAM-4) (Spring et al., 2001, Blood 98, 458-466; Mankelow et al., 2004, Blood 103, 1503-1508). Mutant clones were confirmed by DNA sequence analysis. Domain deletion clones were obtained by PCR of full length Lu cDNA in pIg vector. For Lu domain 3+4+5, 3+4 and 3 primers that contained sense sequence from the start of domain 3 with anti-sense leader sequence (5′-gcc cag gcg gag tat ccc acg gag-3′; SEQ ID NO: 19) and anti-sense sequence from the start of domain 3 and sense leader sequence (5′-ctc cgt ggg ata ctc cgc ctg ggc-3′; (SEQ ID NO: 20) along with either sense and anti-sense pIg or anti-sense end of domain 3 (5′-act tgg gat cca ctt acc tgt cag ctc cag cgt-3′; SEQ ID NO: 21) or 4 (5′-act tgg gat cca ctt acc tgt tgg cga gcc ttg-3′; SEQ ID NO: 22) primers (containing an inframe BamHI restriction site) were used to amplify the leader sequence and either domains 3-5, domains 3-4 or just domain 3. In a second PCR the leader sequence was annealed to each domain deletion PCR product by means of the overlap before being restricted and ligated into pIg vector. For Lu domain 1+2, 1+2+3 and 1+2+3+4 one PCR was performed using sense pIg primer with anti-sense end of domain 2 (5′-act tgg gat cca ctt acc tgt gtg ctc cgt ggg-3′; SEQ ID NO: 23) 3 or 4 primers (as above) the products being inserted into pIg vector. Two types of domain 1−2−3 proteins where used, D1+2+3a was made for this paper, D1+2+3b was constructed as described in Parsons et al. (1997). Lu mutant and domain deletion constructs were fully sequenced and native, mutant and domain deletion Lu gpFc fusion proteins (Lu gpFc) were expressed in COS-7 cells as described previously (Simmons, 1993, Cloning cell surface molecules by transient expression in mammalian cells. Cellular interactions in development: A practical approach, IRL press, Oxford, UK) and purified from Culture supernatant using protein A-Sepharose. An ELISA utilising a known standard IgG curve was used to determine the concentration of the Lu gpFc proteins.

### Western Blotting

Purified Fc fusion proteins were separated on 7.5% SDS polyacrylamide gels and transferred to PVDF membrane as described in Sambrook et al. (1989, Molecular Cloning: A laboratory Manual. Cold Spring Harbor Press, Cold Spring Harbor, N.Y., USA). Membranes were probed with Lu gp antibodies BRIC 108, BRIC 221 and BRIC 224 (Parsons et al., 1987; Parsons et al., 1995) or an HRP linked anti human Fc polyclonal (Sigma, Dorset, UK).

### Lu gp Binding LM511/521 ELISA

All washes and protein dilutions were performed in PBS 0.2% BSA, all protein dilutions were added in 50 μl and incubations were at 37° C. with shaking for one hour unless stated otherwise. Immulon-4 96 well plates (Dynes Technologies, West Sussex, UK) were coated with 0.25 μg/well goat-antihuman-Fc (Jackson ImmunoResearch, Cambridgeshire, UK) in 0.1M bicarbonate buffer, pH 9.6 for 24 hours at 4° C. After three washes 0.05 nM of native or mutated Lu gpFc protein was added and the plate incubated. After one wash the plate was blocked for 30 minutes at room temperature in PBS 0.2% BSA 5% human AB serum followed by one wash. LM511/521 (Chemicon, Hampshire, UK) at 5 nM was added and the plate incubated. After three washes a 1 in 100 dilution of rabbit anti Laminin (Sigma, Dorset, UK) was added and the plate incubated and after three washes a 1 in 1000 dilution of a horse radish peroxidease (HRP) linked swine anti-rabbit (DAKO, Cambridgeshire, UK) was added to the plate, incubated and washed a further three times. The plate was developed using 3,3′,5,5′-Tetramethylbenzidine and 3% H₂O₂ in 0.1M acetate/citrate buffer pH6, stopped with 2M H₂SO₄ and read at 450 nm. The ELISA was controlled both positively by coating wells with 5 nM LM511/521 and negatively with the addition of 5 nM LM511/521 to captured Muc18 Fc fusion protein (a gift from Dr Simmons). In assays performed using different buffers, after the Lu gpFc had been added to the plates, control wells were incubated with the different buffers and LM511/521 added in PBS-BSA whereas other wells were incubated with LM511/521 in the different buffers.

### Surface Plasmon Resonance Assays

All assays were performed at 25° C. using a Biacore X, a CM5 chip with protein A (Sigma, Dorset, UK) immobilised on its surface, a flow rate of 30 μl per minute and with phosphate buffered saline pH 7.4 containing 0.05% Tween 20. Re-generation of the chip was performed with 0.1M glycine pH 2. Native or mutant Lu gpFc was captured onto the chip until a change of 40 response units (RU's) was observed (0.25 pm). LM511/521 was added in a 100 μl injection of 10 nM (1 pm).

### Sidec Tomographical Reconstruction of Lu gp and LM511/521

Three samples, A, B and C, containing an equimolar mixture of Lu gpFc and LM511/521, just Lu gpFc and a mixture of Lu gp and BRIC 108 respectively were analysed by Sidec Technologies AB, Torshamnsgatan 28A, SE-164 40, Sweden (www.Sidec.com) using Protein Tomography™. Protein Tomography™ is a three dimensional imagining tool for studying protein conformations. It uses low-dose electron tomography in combination with refinement algorithms to reconstruct individual macromolecules and complexes (Skoglund et al., 1986, Nature 319, 560-564; Gherardi et al., 2006, Proc. Natl. Acad. Sci. USA 103, 4046-4051; Wartiovaara et al., 2004, J. Clin. Invest 114, 1475-1483).

### Erythroblast Culture

CD34 positive cells were purified from human peripheral blood by positive selection using the MiniMACS magnetic beads system (Miltenyi Biotec Ltd, Surrey, UK), according to the manufacturer's instructions. Suspension cultures were maintained in Stem Span medium (StemCell Technologies, London, UK) containing IL-3 (10 ng/mL), SCF (100 ng/mL) (R&D Systems, Oxfordshire, UK), erythropoietin (3 U/mL) and Prograf (0.1 ng/mL) (Roche, East Sussex, UK), low density lipoprotein (20 mg/ml) (Calbiochem Merck, Nottingham, UK). Cultures were maintained at a cell concentration of between 2 and 10×10⁵ and at 37° C. 5% CO₂ for up to 21 days. Antigen expression at various time points was measured by flow cytometry on a FACSCalibur (Becton Dickinson, Oxfordshire, UK) as described in Smythe et al. (1996, Blood 87, 2968-2973).

### Cell Adhesion Assay

LM511/521 or Laminin 1 (Sigma, Dorset, UK) was coated onto 96 well immulon 4 plates in PBS at 4° C. for 18 hours. Erythroblasts at various stages of development were suspended in Iscoves Modified Eagles medium containing 5% human AB serum (assay buffer) and fluorescently labelled by incubation with 10 μg/ml 2′, 7′-bis(2-carboxyethyl)-5(6)-carboxyfluorescein acetoxymethyl ester (BCECF-AM Sigma, Poole, UK) for 15 minutes at 37° C. After 3 washes in assay buffer erythrocytes were added at 10⁵ cells per well in assay buffer and allowed to adhere at 37° C. for 15 minutes. Fluorescence was measured before and after the unbound cells were washed off the plate and the percentage bound erythrocytes quantitated. A stable K562 transfectant expressing Lu gp (Parsons et al., 1997) binding to LM511/521 and Laminin 1 were included as a control in each assay.

### Results

### Construction of an Homology Model for Amino-Terminal Domains 1-5 of the Lu gp.

Previous work has shown that the LM511/521 binding site on Lu gp resides within the first three Ig domains or alternatively on domain 5. In order to identify the precise location of the binding site we constructed a molecular model of the five extracellular Ig domains using known structures of V-set (domain 1 and 2) and I set (domains 3, 4 and 5) IgSF domains (FIG. 1a). The categorization in this study of domains 3, 4 and 5 as I-set IgSF domains constituted a reclassification of domains 3, 4 and 5, which had originally been classified as C2-set. The original classification of these three domains (Parsons et al., 1995) was prior to the first description of the I-set (Harpaz & Chothia, 1994, J. Mol. Biol. 238, 528-539), which was so named as it is structurally intermediate between the V and C sets. The sequences of domains 3, 4 and 5 matched I-set sequence patterns significantly better than C2-set patterns. In the final model 89% of residues lie within the most favored regions of the Ramachandran plot and the quality of all stereochemical parameters is equal to or better than that expected of an experimental structure of 2.0 Å resolution (Laskowski et al., 1993). The modeling was hindered due to the low sequence identity between Lu gp and the template structures (18%). However, as IgSF domains are well characterized the model may be expected to be a good estimate of the true structure within the beta-sheet core of each domain. The exact conformation of the inter-strand loop regions and the relative orientations of the domains are necessarily more speculative due to the lack of template structures with high sequence identity. The model reveals a compact organisation at the interface of domains 1 and 2, 3 and 4 and 4 with 5. Domains 3, 4 and 5 are predicted to take the form of a rod-like structure. The interface between domains 2 and 3 comprises a linker or hinge region of 6-8 amino acids suggesting flexibility in the structure at this point. Nineteen inherited antigens have been described on the Lu gp of human erythrocytes. Each antigen results from a single nucleotide substitution. Antigens are located on four of the five Ig domains and the fact that all the relevant residues are surface exposed in the model suggests that the orientation of amino-acids in each domain is accurate (FIG. 1b). Of particular interest is the LU12 antigen which can result from deletion of R3 and L4 or the substitution R109Q. Though separated in the linear sequence these residues are adjacent in the molecular model (FIG. 1b).

Salt and pH Dependence of the Interaction of LM511/521 with Lu gp.

We reasoned that a negatively area in Lu gp was a possible site for LM511/521 binding on the Lu gp and investigated the effect of pH (4-11) and high salt (1M NaCl) on the interaction of Lu gp with LM511/521 in an ELISA. The results demonstrated inhibition of Lu gp/LM511/521 interaction in the presence of high salt and at a pH above 10 or below 5 (FIG. 2). Controls exposing Lu and LM511/521 coated to 96 well plates showed that neither protein were permanently denatured by the various buffers as Lu gpFc could still bind to LM511/521 and LM511/521 was still recognised by the anti-Laminin antibody used in these assays (FIG. 2). Inhibition in high salt concentrations is consistent with charged or polar interactions between the two molecules. The fact that the interaction occurs at physiological pH but not at high (over pH10) or low (under pH5) suggests it is mediated by acidic and basic amino-acid residues. It is known that there are numerous areas of positive charge on Laminin LG's as a result of large numbers of lysine and arginine residues so it would seem likely that these are being affected at high pH and, by inference, any interacting aspartic and glutamic acid residues affected at low pH would be present on Lu gp.

### Site-Directed Mutagenesis of Acidic Residues on Domains 2 and 3 of Lu gp.

Analysis of the homology model of Lu gp allowed us to target surface-exposed clusters of aspartic and glutamic acid residues for mutational analysis. The majority of the residues selected for mutagenesis are conserved or identical in the mouse Lu gp homologue (Table 2) and were mutated either individually or as pairs as described in the methods section. Each mutant Lu gp was examined by western blotting with monoclonal anti-Lu (ERIC 108, BRIC 221 and BRIC 224) to ensure that the expressed protein was of the expected size and had sufficient structural similarity to the native protein to maintain the three antibody epitopes (BRIC 224 FIG. 3a, BRIC 108 and 221 not shown). Each mutant protein showed an identical band pattern to that of the native protein with a band corresponding to a Lu gpFc dimer at 238 kDa (FIG. 3a). The epitopes for BRIC 108, 221 and 224 are structural and destroyed by reducing conditions. As each mutant protein is recognised by all three antibodies it demonstrates that the mutation has not caused a catastrophic change in the overall structure of Lu gpFc. A western blot under reducing conditions using anti human Fc shows Lu gpFc in its monomeric form and that all the monomeric forms of the mutant proteins also are of the same size (108 kDa) (FIG. 3b). The apparent molecular weights of monomers and dimers (FIGS. 3a and b) compare well to the sizes of 113 and 226 kDa predicted from composition analysis.

TABLE 2
Comparison of sequence in human and
murine Lu gp at the sites of mutation.

| Lu gp | | | |
|---|---|---|---|
| mutation | Domain | Human Lu gp | Murine Lu gp |
| E132A/D133A | 2 | SVMEDSAQ | SVMDQFAQ |
| | | (SEQ ID NO: 24) | (SEQ ID NO: 25) |
| E180A | 2 | RTVREASG | RTVREASG |
| | | (SEQ ID NO: 26) | (SEQ ID NO: 27) |
| D198A/D199A | 2 | LRKDDRDA | LHKDDRDA |
| | | (SEQ ID NO: 28) | (SEQ ID NO: 29) |
| E234A | 2 | YPTEHVQF | YPTEHVEF |
| | | (SEQ ID NO: 30) | (SEQ ID NO: 31) |
| E269A | Domain | PSPEYTLF | PSPEYSFF |
| | 2-3 | (SEQ ID NO: 32) | (SEQ ID NO: 33) |
| | Hinge | | |
| E280A | 3 | DEQEEVLN | GTQEEQLN |
| | | (SEQ ID NO: 34) | (SEQ ID NO: 35) |
| E281A | 3 | DEQEEVLN | GTQEEQLN |
| E309A | 3 | EDYDAADD | EDYDADEE |
| | | (SEQ ID NO: 36) | (SEQ ID NO: 37) |
| D310A | 3 | EDYDAADD | EDYDADEE |
| D312A | 3 | EDYDAADD | EDYDADEE |
| D315A | 3 | EDYDAADD | EDYDADEE |
| D316A | 3 | EDYDAADD | EDYDADEE |

Human Lu gpis highly homologous to its murine counterpart. Shown are the 15 mutations to Lu gp constructed in this study along with the corresponding residue that is present in the murine homologue at the same position.

The LM511/521 binding properties of mutant Lu proteins were examined using an ELISA type assay (as described above). The results showed that the D312A mutation causes a severe reduction in binding (FIG. 3c). Two further mutations, E309A and D310A show a marked decrease in adhesion to LM511/521 whereas E132A/D133A, D198A/D199A, E269A, D280A and D316A caused only a slight decrease in binding (FIG. 3c). The other mutant proteins showed the same or similar levels of adhesion to LM511/521 to that obtained with native Lu gpFc protein (FIG. 3c). There is a cluster of positively charged residues on domain 2 comprising of R179, R194 and R196 that depending on the orientation of Domain 2 with 3 could potentially interact with E309, D310 and D312, however, Lu gpFc containing a R179A, R194A or R196A mutation bound LM511/521 as native Lu gpFc (data not shown).

The mutant proteins were also assayed for LM511/521 binding using a Biacore X (FIG. 4). A sensorgram of native Lu gpFc adhering to LM511/521 is shown in FIG. 4a. The sensorgrams for each mutation when compared with that of the native protein showed that the effect caused by each mutation when assayed on the Biacore is similar to that observed when assayed by ELISA (FIG. 4b, Table 3). Data from the Biacore assays show that mutations that affect LM511/521 binding slow the rate at which LM511/521 binds to Lu gpFc (FIG. 4c). The rate at which LM511/521 dissociates is not affected by the various mutations (FIG. 4d). When compared to our earlier study (Parsons et al., 2001) the speed of dissociation of Lu gpFc and LM511/521 when observed in the Biacore assays is somewhat slower in the present experiment. In Parsons et al. (2001), the assays were performed using an Iasys optical biosensor as opposed to a Biacore and the commercial source of LM511/521 was different from that used in this study. Therefore it is difficult to make direct comparisons between the two studies.

TABLE 3
Comparison between the ELISA and Biacore assays of the
mutant Lu gp. Binding of LM511/521 to the different mutant Lu
gp's ability to bind to LM511/521 was assayed using an ELISA
based assay and a BiacoreX. The table shows the % binding of
LM511/521 relative to that of native Lu gpFc observed by ELISA
and the total Δ RUs observed in the Biacore. The mutants are
listed according to their rank on the level of binding on the
Biacore.

| | Biacore | Biacore | ELISA | ELISA |
|---|---|---|---|---|
| | (Δ RUs) | Rank | (% of native Lu gpFc) | Rank |
| Native Lu gpFc | 114.65 | 2 | 100 | 2 |
| D315 | 116.35 | 1 | 93.2 | 4 |
| D281A | 100.4 | 3 | 97.3 | 3 |
| D280A | 99.1 | 4 | 73.0 | 7 |
| E234A | 85.0 | 5 | 90.7 | 5 |
| D316A | 79.8 | 6 | 75.0 | 6 |
| E180 | 78.3 | 7 | 103.9 | 1 |
| D269 | 73.8 | 8 | 63.2 | 9 |
| D198A-D199A | 59.7 | 9 | 49.0 | 10 |
| E132A/D133A | 57.3 | 10 | 69.4 | 8 |
| D309A | 44.9 | 11 | 40.4 | 11 |
| D310A | 38.5 | 12 | 26.4 | 12 |
| D312A | 4.4 | 13 | 20.7 | 13 |

The results demonstrate that E132A/D133A, D198A/D199A, E269A, D316A and to a much greater extent E309A, D310A and D312A, inhibit LM511/521 binding to Lu gp by preventing the interaction of the two proteins. The location and importance of each amino-acid is indicated on the molecular model of Lu gp in FIG. 5. A more recent X-ray crystal structure of Lutheran glycoprotein domain 2 reveals that the position of E180 lies further away from the domain 2−3 boundary than shown in FIG. 5 and that this residue is closer to the domain 1−2 boundary. The positions of other amino acids on domain 2 such as D133, E133, D199 and D198 are similar to those shown in FIG. 5, and are close to the domain 2−3 boundary.

LM511/521 Binding to Lu gpFc Domain-Deletion and Domain 2−3 Hinge Mutants

The results of site-directed mutagenesis described above clearly identify residues E309, D310 and D312 on domain 3 as vital for adhesion of LM511/521 and indicate that a further 8 acidic residues on domains 2 and 3 may play a role in this interaction. We extended these studies by constructing additional domain-deleted proteins in order to further understand the contribution of the overall structure of Lu to LM511/521 adhesion. Six different Lu proteins were made containing domains 1 and 2 (D1+2) alone; domains 1, 2 and 3 (D1+2+3a or b); domains 1, 2, 3 and 4 (D1+2+3+4); domains 3, 4 and 5 (D3+4+5); domains 3 and 4 alone (D3+4) and domain 3 alone (D3) (FIG. 6a). Western blots using BRIC 224 and BRIC 221 which recognise epitopes on domains 1 and 4 respectively, demonstrated each mutant protein (except the domain 3 only construct) was correctly folded and that all had an apparent molecular weight consistent with the expected domain composition (FIGS. 6b and c). We also attempted to construct a protein containing domains 2, 3, 4 and 5, however, expression of this protein was very low and the resultant protein was not detected on a Western blot with BRIC 221 and degraded rapidly even at 4° C. (not shown). When the domain-deletion mutants were examined for their ability to bind LM511/521 only proteins containing domain 2 and domain 3, together with the hinge region between them supported LM511/521 adhesion (FIGS. 6d and e). Despite containing key residues involved in binding LM511/521 Lu gp proteins containing domain 2 without domain 3, and more significantly, domain 3 without domain 2 are unable support binding of LM511/521.

The protein containing domains 1 to 3 constructed for this example (D1+2+3a) did not adhere at all to LM511/521 in an ELISA or a Biacore assay. However, a fresh preparation of a similar protein but with a run of thirteen extra amino-acids linking Lu gp to the Fc piece of the fusion protein (D1+2+3b) (Parsons et al., 2001) adhered to LM511/521, but exhibited 68% the level of binding in the ELISA and only 42% in the Biacore assay as the native Lu gp or the D1+2+3+4 protein (FIGS. 6d and e).

The observation that domains 2 and 3 are both essential for LM511/521 binding raised the question of the importance of the linker or hinge sequence between these domains. We constructed two Lu gpFc mutants in which residues in the hinge were replaced with a proline (T233P and H235P) and a third mutant that has three residues removed from the hinge (Δ233-235) (FIG. 6a). Western blotting confirmed these molecules were folded correctly (FIGS. 6b and c). LM511/521 binding by mutant T233P was markedly reduced and that of H235P and Δ233-235 was abolished (FIGS. 6d and e). These data are consistent with an essential role for the hinge region in allowing domains 2 and 3 to take up a conformation capable of binding LM511/521.

Taken in their entirety these mutagenesis data suggest the primary binding site for LM511/521 is defined by negatively charged residues on domain 3 and to a lesser extent domain 2 but the appropriate presentation of domains 2 and 3 for LM511/521 binding is influenced by domains 1 and 4 and most importantly by the hinge between domains 2 and 3.

Imaging of Lu gpFc-LM511/521 Interaction by Electron Tomography

In order to explore the nature of Lu gp-LM511/521 binding further. Lu gpFc alone and bound to LM511/521 or BRIC 108 was examined using electron tomography. Sidec Protein Tomography' is a new three-dimensional imaging tool for analysis of protein conformation. The method uses low-dose electron tomography in combination with refinement algorithms to reconstruct individual proteins and macromolecular complexes in biological samples. Shown in FIG. 7a are images of LM511/521 bound to Lu gpFc.

Three-dimensional reconstructions were generated and objects with a molecular weight in the range 750-1100 kDa visualised. The results indicated that approximately 50-60% LM511/521 molecules bound Lu gpFc and that most of these complexes contained dimeric Lu gpFc. Inspection of FIG. 7a reveals binding of dimeric Lu gpFc to the C-terminal domain. In order to determine those parts of Lu gpFc in direct contact with LM511/521 individual Lu gpFc monomers were identified and orientation of the domains within the Lu gpFc molecule determined by analysis of a complex between Lu gpFc monomer and monoclonal antibody BRIC 108 which recognises an epitope of domain 1 (FIG. 7b). These experiments allowed the assignment of domains shown in FIG. 7c. These data are consistent with the Lu gp binding site being contained in the negatively charged region on domains 2(V2) and 3(I1) and further suggest that domain 1(V1) folds back when the binding site is revealed. These observations could be relevant to evidence indicating an indirect role for domain 1 in creating the LM511/521 binding site.

As the Protein Tomography™ evidence suggested that Lu gp could potentially exist in a folded confirmation we processed our model through the PatchDock web server (http://bioinfo3d.cs.tau.ac.il/PatchDock/) to explore how domains 1 and 2 could potentially interact with domains 3, 4 and 5. FIG. 7d and e shows the most viable orientation of the Lu gp domains as laid down by the structural constraints of the model and in particular the hinge region between domains 2 and 3. As the orientation of the two Fc domains in relation to the 5 Lu gp domains and the size and structure of at least 5 N-linked glycans within Lu gpFc are unknown any attempt to fit the Protein Tomography™ data with our model would be highly speculative.

### Lu gp is the Only LM511/521 Binding Protein on Human Erythrocytes.

Lu gp is found in many tissues at sites of cell-matrix interactions where it colocalises with Laminin-binding integrins and the tetraspanin CD151. Lu gp is also expressed on the surface of erythroblasts late on in their development, at the orthochromatic erythroblast stage. Erythroblasts at this stage of development lack Laminin-binding integrins with the only integrin present being the non-Laminin binding alpha 4 beta 1. Late stage erythroblasts adhere to LM511/521 and this adhesion correlates with expression of Lu gp on their surface (FIG. 8). In addition erythroblasts from a culture developed from the peripheral blood of two individuals (Lu(a-b-) of the In(Lu) genetic background) whose erythroid cells do not express Lu gp on their surface do not adhere to LM511/521 (FIG. 8). These results indicate that Lu gp is the only LM511/521-binding protein found on late stage erythroblasts.

### DISCUSSION

The interaction between LM511/521 and Lu gp involves positively charged basic residues on the LM511/521 and areas of negative charge on its ligand Lu gp. The interaction is inhibited by high salt (FIG. 2; (15)) and by high (>10) and low (<5) pH. Since there are areas on Laminin LG domains that consist mainly of arginine and lysine residues and these would be neutral over pH10 it seemed likely that negatively charged residues on Lu gp could be responsible for hindering the interaction with LM511/521 at low pH.

Inspection of the three-dimensional model of extracellular Ig domains 1-5 of Lu gp revealed a negative patch at the junction of domains 2 and 3. This patch is conserved in the murine homologue that also binds to LM511/521 (Parsons et al., 2001). When negatively charged residues in the patch were mutated to alanine and the mutant proteins assessed for LM511/521 binding by ELISA and Biacore assay residue D312 and, to a lesser extent, E309 and D310 were identified as critical residues (Table 3). Other negatively charged residues in the vicinity of these three also had slight effects on binding. It is interesting to note that when the positions of all the mutations made in this study are mapped to the molecular model of Lu gp and coded for severity of effect on LM511/521 binding, they appear to form concentric rings around D312 (FIG. 5). We submit that the negative charge on D312 interacts with a positively charged amino-acid on LM511/521 and that this charged interaction is the primary determinant of adhesion between the two molecules. The other acidic amino-acids identified here could potentially produce a localised negative charge on Lu gp around D312 and facilitate docking by interaction with other positive charges on LM511/521. The close homology between the sequence of human and murine Lu gp suggests it is likely that murine Lu gp and other Lu gps have a similar structure to their human counterpart and binds LM511/521 in an identical manner. The interaction between Lu gp and LM511/521 is very strong (kₐₛₛ=1.6±0.09×10⁶ M⁻¹ s⁻¹/k_{diss}=1.26±0.01×10⁻² s⁻¹, K_{D}=7.9±0.47 nM) when compared with other CAMs and their ligands. The fact that it is a result of the interaction between certainly one and potentially three negatively charged amino acids on Lu gp and an area of concentrated positive charge on LM511/521 would account for this strong attraction between the two proteins.

In the present study we compared two constructs containing domains 1, 2 and 3 and found that one construct bound LM511/521 and one did not. There were only minor differences between the two proteins, one ended L³⁵⁴EL-Fc (D1+2+3a) and the other, L³⁵⁴EL-RVAYLNSPQTSQA-Fc (D1+2+3b; SEQ ID NO: 38) (as described Parsons et al., 2001). Despite both proteins containing all that is required for LM511/521 adherence and seemingly being correctly folded only the D1+2+3b construct binds LM511/521. These results suggest the Fc portion is restricting the structure of the D1+2+3a protein and affecting its ability to bind LM511/521. In addition, we found that a construct of domains 1, 2, 3 and 4 was as effective in binding LM511/521 as the construct with all five domains but the D1+2+3b construct was not as effective as the D1+2+3+4 construct. These data suggest the orientation of domains 1 and 4 relative to domains 2 and 3 also influence the optimal conformation for LM511/521 binding.

Inspection of the molecular model of Lu gp reveals a hinge of approximately ten amino-acids between domains 2 and 3. This suggests that unlike the junctions between other domains, that between domains 2 and 3 is very flexible. Consequently, there is potential for Lu gp to occupy a number of different structural conformations between fully elongated and bent at the hinge. We have shown that by placing a proline in this hinge region (T233P and H235P) or by removing 3 amino-acids (Δ233-235) and shortening the hinge Lu gp binding to LM511/521 is totally abolished. These data could be interpreted as indicating residues T233, E234 and H235 are directly involved in binding to LM511/521. However, this seems unlikely since mutation E234A has only a minor effect on binding (FIGS. 3c iv and 4b).

To further explore the structure of Lu gp we obtained images of a Lu gpFc using Sidec Protein Tomography™. Images of Lu gpFc bound and unbound to LM511/521 are shown in FIG. 7. The images portray Lu gpFc in a bent conformation (FIG. 7c) both when free in solution and when bound to LM511/521. The part of LM511/521 to which Lu gpFc binds is readily identified as the C terminal alpha 5 globular domain. Experiments (FIG. 7b) were also carried out with Lu gpFc bound to a monoclonal antibody (BRIC 108) which recognises an epitope on Lu domain 1. These experiments suggested the assignment of Lu domains shown in FIG. 7c and that Lu gpFc has a structure which exposes the LM511/521-binding negatively charged region on domains 2 and 3 with domain 1 folded back and aligned with the rod-like domain 4, 5 backbone. This conformation could potentially result in all the aspartic and glutamic acids shown to be involved in binding LM511/521 being spatially very close leading to a large negatively charged area on the surface of Lu gp involved in LM511/521 adhesion (FIG. 7d). The tomographical data are compatible with the molecular model and mutation analysis.

One can envisage two possible consequences of Lu gp binding to LM511/521. Firstly, LM511/521 binding could cause a structural change in Lu gp associated with “outside in” signalling. Secondly, intracellular signals could influence the conformation of the extracellular domain of Lu gp and modulate LM511/521 binding by “inside out” signalling. Our results demonstrate that the binding site for LM511/521 is defined by negatively charged residues on domains 2 and 3 and that the hinge region between these domains is critical for LM511/521 binding. In this context it is interesting to note that the T233P mutation causes a reduction in adhesion to LM511/521 that is much less marked than the H235P mutation and the Δ233-235 deletion. Since the orientation of domains 1 and 2 relative to 3, 4 and 5 is determined by the angle the hinge leaves domain 3 and since H235 is closer to domain 3 than T233, the H235P mutation would be expected to have a greater effect on the orientation of domains 1 and 2 relative to domain 3 and thus a greater effect on adhesion to LM511/521. Results obtained with the two D1+2+3 constructs and the D1+2+3+4 construct further suggest that domain 4 influences the structure of the binding site. These observations are consistent with data from electron tomography indicating that LM511/521 binding is effected by a conformation of Lu gp in which domain 1 folds back and aligns with domain 4 in order to expose the critical residues for binding (FIGS. 7a, b, d and e).

Our finding that Lu gp is the only LM511/521 binding molecule on late-stage human erythroid cells suggests that the erythrocyte may provide a useful model for exploring the functional consequences of Lu gp-LM511/521 binding. It is known that LM511/521 is expressed in the bone marrow sinusoidal endothelium raising the possibility that the role of Lu gp in erythropoiesis is in trafficking mature erythrocytes out of the bone marrow. We consider that erythroblastic islands consisting of late stage differentiated erythroid cells migrate to the sinusoid where adhesion between Lu gp and LM511/521 is stronger than that between the erythroblast/reticulocyte and macrophage within the island causing release of the erythroid cells from the island. Erythroid cells bound to LM511/521 then migrate across the sinusoidal endothelium resulting in release of erythrocytes into the vascular system. In addition, there is evidence the interaction between Lu gp and LM511/521 is of importance in mediating vaso-occlusion at sites of inflammation during sickle cell crisis (see Parsons et al., 1999, Baillieres Best. Pract. Res. Clin. Haematol. 12, 729-745). In human erythrocytes the cytoplasmic domain of Lu gp is known to be capable of interacting with spectrin, a component of the red cell skeleton. It is also reported that intercellular signalling mechanisms can modulate phosphorylation of the Lu cytoplasmic domain and consequently increase or decrease Lu gp's adherence to LM511/521. These data suggest a role for Lu gp in modulating adhesion between cells and extracellular matrix rather than maintaining the structure of a tissue, a role which is more likely fulfilled by integrin/CD151 interactions with LM511/521. The occurrence of apparently healthy rare individuals with defects in the human Lutheran gene preventing expression of Lu gp in any cells or tissues is consistent with this suggestion.

Our findings provide clear evidence that Lu gp interaction with LM511/521 is mediated by a negatively charged patch on Lu gp extracellular domain 3 in a manner analogous to that occurring for the other ligands of Laminin, heparin and α-dystroglycan. In this case, the negatively charged patch is composed of glutamic and aspartic acid residues rather than sulphated sugars. Our results further suggest that the LM511/521 binding site on Lu gp is located at a flexible hinge region in an otherwise rigid structure and that amino-terminal domains 1 and 2 may fold back to expose the binding site for LM511/521 binding.

### Example 2

A pharmacophore model of the LM511/521 binding site on Lu gp is developed using the Tripos SYBYL molecular modeling suite of programs (Tripos, St Louis, Mo., US) based on molecular data relating to the binding site as outlined above. The pharmacophore model is used in silico to screen a library for molecules which are predicted to interact with the binding site.

### Example 3

In order to verify a molecule identified in Example 2, the molecule is added at various relevant concentrations in the ELISA method described in Example 1 at the same time as the Lu gpFc. The results will show whether or not the molecule is able to bind to and/or inhibit Lu gp.

Using the Biacore system described in Example 1, an antagonist or enhancer molecule can be injected at the same time as laminin 511/521 to calculate how the molecule affects the on/off rate of laminin 511/521 binding to Lu gp.

The Kaul system involving intravital microscopic observations of human sickle cells flowing through rat mesocecum vasculature may also be used to investigate molecular interactions. An antagonist or enhancer molecule of the Lu gp laminin 511/521 binding site is added at the same time as human sickle cells to investigate the effects on the interaction between Lu gp on the sickle cells and laminin 511/521, as described for monoclonal antibodies (Kaul et al., 2000, Blood 95, 368-374) and peptides (Kaul et al., 2006, Am J Physiol Cell Physiol 291, C922-30).

### Example 4

A sickle cell patient (or other “at risk” patient) may be administered, for example over a continuous period, with an antagonist molecule which inhibits or prevents binding of Lu gp to laminin 511/521. During a crisis phase or coinciding with the onset of vascular damage, binding of Lu gp to any exposed laminin 511/521 is diminished or prevented, thereby reducing or eliminating vaso-occlusion or thrombotic events.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1-32. (canceled)

33. An isolated polypeptide up to 550 amino acid residues in length comprising a laminin ligand binding site formed by the peptide sequence EDX₁D (SEQ ID NO: 1), where X₁ is any amino acid, in which the polypeptide is a functional fragment of Lu gp or a homologue or variant of the functional fragment, excluding the polypeptides having any of the sequences SEQ ID NOs: 11-13 or 15-18.

34. The isolated polypeptide according to claim 33, comprising the peptide sequence EDX₁DAAX₂X₃ (SEQ ID NO: 2), where X₂ and X₃ are any amino acid.

35. The isolated polypeptide according to claim 33, in which X₁ is selected from the group consisting of tyrosine (Y), glutamic acid (E) and aspartic acid (D).

36. The isolated polypeptide according to claim 34, in which X₂ and/or X₃ are each independently selected from the group consisting of glutamic acid (E) and aspartic acid (D).

37. The isolated polypeptide according to claim 35, in which X₂ and/or X₃ are each independently selected from the group consisting of glutamic acid (E) and aspartic acid (D).

38. The isolated polypeptide according to claim 33, the polypeptide comprising the sequence EDYDAADD (SEQ ID NO: 3) or EDYDADEE (SEQ ID NO: 4).

39. A method for identifying a molecule which is an antagonist of a Lu gp binding site for a laminin isoform having an α5 chain (for example laminin 511 or laminin 521), in which the method comprises identifying, on the basis of biological activity and/or structural and/or chemical properties and/or electronic environment of the binding site and/or the molecule, a molecule which replicates and/or interacts with a polypeptide according claim 33.

40. The method according to claim 38, in which the method is performed in silico.

41. A recombinant polypeptide having at least 40% sequence identity to wild-type mature human Lu gp, in which an amino acid in the polypeptide corresponding to residue 312 in mature human Lu gp is other than aspartic acid, such that the polypeptide has impaired affinity for a laminin isoform comprising an α5 chain (for example, laminin 511 or laminin 521) compared with the corresponding wild-type Lu gp.

42. The polypeptide according to claim 41, in which an amino acid in the polypeptide corresponding to residue 235 in mature human Lu gp is other than histidine.

43. The polypeptide according to claim 41, in which an amino acid corresponding to residue 309 in mature human Lu gp is other than glutamic acid and/or an amino acid corresponding to residue 310 in mature human Lu gp is other than aspartic acid.

44. The polypeptide according to claim 41, in which an amino acid corresponding to residue 198, 199 and/or 269 in mature human Lu gp is other than aspartic acid.

45. The polypeptide according to claim 41, in which an amino acid corresponding to residue 133 in mature human Lu gp is other than aspartic acid or glutamine.

46. The polypeptide according to claim 41, in which an amino acid in the polypeptide corresponding to residue 133, 198, 199, 235, 269, 309, 310 and/or 312 in mature human Lu gp have been subjected to point mutation and/or deletion.

47. The polypeptide according to claim 41, in which the amino acid corresponding to residue 133, 198, 199, 235, 269, 309, 310 and/or 312 in mature human Lu gp is alanine.

48. A kit for testing binding of a laminin isoform comprising an α5 chain (for example, laminin 511 or laminin 521) with Lu gp or a fragment thereof, comprising an Lu gp polypeptide as defined in claim 33.

49. An isolated polynucleotide encoding a polypeptide according to claim 33.

50. A kit for testing binding of a laminin isoform comprising an α5 chain (for example, laminin 511 or laminin 521) with Lu gp or a fragment thereof, comprising an Lu gp polypeptide as defined in claim 41.

51. An isolated polynucleotide encoding a polypeptide according to claim 41.
